(19) United States
(12) Patent Application Publication
(10) Pub. No.: US 2020/0048618 A1
(43) Pub. Date: Feb. 13, 2020

(54) Cell

(71) Applicant: AUTOLUS LIMITED, London (GB)

(72) Inventors: Shaun Cordoba, London (GB); Evangelia Kokalaki, London (GB); Martin Pulé, London (GB); Simon Thomas, London (GB); Shimobi Onuoha, London (GB)

(21) Appl. No.: 16/606,000

(22) PCT Filed: Apr. 17, 2018
(86) PCT No.: PCT/GB2018/050997

§ 371 Date: Oct. 17, 2019

(30) Foreign Application Priority Data
Apr. 18, 2017 (GB) 1706123.5
Dec. 6, 2017 (GB) 1720343.1

(51) Int. Cl. C12N 9/12; C07K 14/73; C07K 14/705; C07K 16/28; A61K 35/17; A61P 35/00; C07K 14/725; C07K 14/71

(52) U.S. Cl. CPC C12N 9/12; C12Y 207/10001; C07K 14/70514; C07K 14/70517; C07K 16/2803; A61K 35/17; A61K 38/00; C07K 14/7051; C07K 14/70578; C07K 14/71; C07K 2319/30; C07K 2319/33; C07K 2319/03; A61P 35/00

(57) ABSTRACT

The present invention relates to a cell which coexpresses a first chimeric antigen receptor (CAR) and a second CAR, wherein the first CAR comprises a phosphorylation amplifying endodomain and wherein the second CAR comprises an activating endodomain.

### FIELD OF THE INVENTION

The present invention relates to a cell expressing a chimeric antigen receptor (CAR).

### BACKGROUND TO THE INVENTION

Chimeric Antigen Receptors (CARs)

A number of immunotherapeutic agents have been described for use in cancer treatment, including therapeutic monoclonal antibodies (mAbs), bi-specific T-cell engagers and chimeric antigen receptors (CARs).

Chimeric antigen receptors are proteins which graft the specificity of a monoclonal antibody (mAb) to the effector function of a T-cell. Their usual form is that of a type I transmembrane domain protein with an antigen recognizing amino terminus (binder), and a transmembrane domain connected to a compound endodomain which transmits T-cell survival and activation signals, as shown in FIG. 1(a). The endodomain may comprise one or more activating endodomains, such as 4-1BB or OX40, which transmit survival signals. Even more potent generation CARs have been described which have endodomains capable of transmitting activation, proliferation and survival signals, as shown in FIGS. 1(b)-(d).

The most common form of these molecules are fusions of single-chain variable fragments (scFv) derived from monoclonal antibodies, which recognize a target antigen, fused via a trans-membrane domain to a signalling endodomain. Such molecules result in activation of the T-cell in response to recognition by the scFv of its target. When T cells express such a CAR, they recognize and kill target cells that express the target antigen. Several CARs have been developed against tumour-associated antigens, and adoptive transfer approaches using such CAR-expressing T cells are currently in clinical trial for the treatment of various cancers.

Various CARs have been tested in vitro and in vivo trials, as summarised in Table 1 below.

TABLE 1

| Target antigen | Associated malignancy |
|---|---|
| α-Folate receptor | Ovarian cancer |
| CAIX | Renal cell carcinoma |
| CD19 | B-cell malignancies |
| CD20 | Lymphomas and B-cell malignancies |
| CD22 | B-cell malignancies |
| CD30 | Lymphomas |
| CD33 | AML |
| CD44v7/8 | Cervical carcinoma |
| CEA | Breast and colorectal cancer |
| EGP-2 | Multiple malignancies |
| EGP-40 | Colorectal cancer |
| erb-B2 | Colorectal, breast and prostate cancer |
| erb-B 2,3,4 | Breast and others |
| FBP | Ovarian cancer |
| Fetal acetylcholine receptor | Rhabdomyosarcoma |
| GD2 | Neuroblastoma |
| GD3 | Melanoma |
| Her2/neu | Medulloblastoma, osteosarcoma, |
| | Glioblastoma, lung malignancy |
| IL-13R-a2 | Glioma, glioblastoma, medullablastoma |
| KDR | Tumor neovasculature |
| k-light chain | B-cell malignancies |
| LeY | Carcinomas, epithelial-derived tumours |
| L1 cell adhesion molecule | Neuroblastoma |
| MAGE-A1 | Melanoma |
| Mesothelin | Various tumours |
| Murine CMV infected cells | Murine CMV |
| MUC1 | Breast, Ovary |
| NKG2D ligands | Various tumours |
| Oncofetal antigen (h5T4) | Various tumours |
| PSCA | Prostate carcinoma |
| PSMA | Prostate/tumour vasculature |
| ROR1 | Various tumours |
| TAA targeted by mAb IgE | Various tumours |
| TAG-72 | Adenocarcinomas |
| VEGF-R2 | Tumor neovasculature |

However, it has been observed that adoptively transferred T-cells sometimes show limited persistence, expansion and/or proliferation in vivo, due to for example, insufficient signalling, lack of IL2 or differentiation.

Additionally, adoptively transferred T-cells may succumb to inhibitory stimuli within the tumour microenvironment. For example, they may become exhausted, undergo activation induced cell death consequent to over activation, or may cause on-target off-tumour effects. These disadvantages may contribute to a reduced proliferation, expansion and persistence of the CAR T-cell even in the more potent generation CAR shown in FIGS. 1(b) to 1(d).

Other examples of adoptively transferred T-cells showing limited persistence, expansion and/or proliferation may be due to low expression profile or low density of the target antigen on the tumour cell or the specific size of the target antigen. In this respect, large target antigens can inhibit close synapse binding, thereby decreasing the sensitivity of the CAR.

CAR T-cell persistence and activity can be enhanced by administration of cytokines, or by the CAR T-cells producing cytokines constitutively. However, these approaches have limitations: systemic administration of cytokines can be toxic; and constitutive production of cytokines may lead to uncontrolled proliferation and transformation.

There is therefore a need for alternative CAR T-cell approaches, which facilitate proliferation, expansion and persistence of T cells, which are not associated with the disadvantages mentioned above.

### SUMMARY OF ASPECTS OF THE INVENTION

The present inventors have developed a new CAR system which has a boosted capacity to induce T cell signalling, leading to enhanced T-cell activation, proliferation and persistence. The CAR system includes a phosphorylation amplifying endodomain which increases the phosphorylation of key molecules involved in T cell activation signalling pathway. By tipping the balance of the system towards phosphorylation, the threshold necessary for T cell activation to occur is decreased.

Thus, in a first aspect of the invention, the present inventors provide a cell which co-expresses a first chimeric antigen receptor (CAR) and a second CAR, wherein the first CAR comprises a phosphorylation amplifying endodomain and wherein the second CAR comprises an activating endodomain.

The first CAR and the second CAR of the cell may bind to different antigens.

The first CAR and the second CAR of the cell may bind to the same antigen.

The first CAR and the second CAR of the cell may bind to different epitopes on the same antigen.

The first CAR and the second CAR of the cell may bind to the same epitope on the same antigen.

The phosphorylation amplifying endodomain of the first CAR of the cell may comprise the tyrosine kinase domain of a Src family kinase (SRK).

The phosphorylation amplifying endodomain of the first CAR of the cell may comprise the tyrosine kinase domain of a SRK selected from Fyn, Src, Lck, and/or mutated Lck (Y505F).

The phosphorylation amplifying endodomain of the first CAR of the cell may comprise the tyrosine kinase domain of Fyn.

The phosphorylation amplifying endodomain of the first CAR of the cell may comprise the intracellular domain of CD4 or CD8 coreceptor.

The activating endodomain of the second CAR of the cell may comprise CD3-ξ.

The activating endodomain of the second CAR of the cell may comprise CD3-ξ and a co-stimulatory domain. The co-stimulatory domain may, for example, be or comprise one or more of 4-1BB, CD28 and/or OX40.

The or each antigen may be expressed at a low density on the target cell.

The or each antigen may have a relatively long or bulky extracellular domain.

The antigen-binding domain of the first CAR and/or second CAR of the cell may bind to the antigen CD22. CD22 has a very long extracellular portion containing seven immunoglobulin domains. CD22 antigens are expressed during early stages in B-cell maturation. It is these cells that develop into B-cell acute leukaemia.

In a second aspect, the present invention provides a nucleic acid construct comprising a first nucleic acid sequence encoding a first CAR as defined in the first aspect of the invention and a second nucleic acid sequence encoding a second CAR as defined in the first aspect of the invention.

The nucleic acid construct may have the following structure:

AgB1-spacer1-TM1-Pa-coexpr-AbB2-spacer2-TM2-endo

in which

AgB1 is a nucleic acid sequence encoding an antigen-binding domain of the first CAR; spacer 1 is a nucleic acid sequence encoding a spacer of the first CAR;

TM1 is a nucleic acid sequence encoding a transmembrane domain of the first CAR; Pa is a nucleic acid sequence encoding the phosphorylation amplifying endodomain of the first CAR;

coexpr is a nucleic acid sequence enabling co-expression of both CARs

AgB2 is a nucleic acid sequence encoding an antigen-binding domain of the second CAR;

spacer 2 is a nucleic acid sequence encoding a spacer of the second CAR;

TM2 is a nucleic acid sequence encoding a transmembrane domain of the second CAR;

Endo is a nucleic acid sequence encoding the activating endodomain of the second CAR;

which nucleic acid sequence, when expressed in a cell, encodes a polypeptide which is cleaved at the cleavage site such that the first and second CARs are co-expressed at the cell surface.

The sequence enabling co-expression of the two CARs may encode a self-cleaving peptide or a sequence which allows alternative means of co-expressing two CARs such as an internal ribosome entry sequence (IRES) or a second promoter.

The spacers of the first and second CARs may be different. The spacers may be tailored to the antigen or epitope recognised by the CAR so that an appropriate distance is provide for the T-cell:target cell synapse for T cell activation to occur.

Alternative codons may be used in regions of sequence encoding the same or similar amino acid sequences, such as the spacer and/or transmembrane domain of the first and second CARs. This may avoid homologous recombination.

In a third aspect, the present invention provides kit which comprises

(i) a first nucleic acid sequence encoding the first chimeric antigen receptor (CAR) as defined in the first aspect of the invention, which nucleic acid sequence has the following structure:

AgB1-spacer1-TM 1-Pa

in which

AgB1 is a nucleic acid sequence encoding the antigen-binding domain of the first CAR;

spacer1 is a nucleic acid sequence encoding the spacer of the first CAR;

TM1 is a nucleic acid sequence encoding the transmembrane domain of the first CAR; and

Pa is a nucleic acid sequence encoding the phosphorylation amplifying endodomain of the first CAR; and

(ii) a second nucleic acid sequence encoding the second chimeric antigen receptor (CAR) as defined in the first aspect of the invention, which nucleic acid sequence has the following structure:

AgB2-spacer2-TM2-endo

in which

AgB2 is a nucleic acid sequence encoding the antigen-binding domain of the second CAR;

spacer2 is a nucleic acid sequence encoding the spacer of the second CAR; and

TM2 is a nucleic acid sequence encoding the transmembrane domain of the second CAR; and

endo is a nucleic acid sequence encoding the activating endodomain of the second CAR.

In a fourth aspect, the present invention provides a kit comprising: a first vector which comprises the first nucleic acid sequence as defined in the third aspect of the invention; and a second vector which comprises the second nucleic acid sequence as defined in the third aspect of the invention.

The vectors may, for example, be integrating viral vectors or transposon vectors.

In a fifth aspect, the present invention provides a vector comprising a nucleic acid construct according to the second aspect of the invention. The vector may, for example, be a retroviral vector or a lentiviral vector or a transposon vector.

In a sixth aspect, the present invention provides a method for making a cell according to the first aspect of the invention, which comprises the step of introducing a nucleic acid construct according to the second aspect of the invention; a first nucleic acid sequence and second nucleic acid sequence as defined in the third aspect of the invention; and/or a first vector and a second vector according as defined in the fourth aspect of the invention, or a vector according to the fifth aspect of the invention, into a cell.

The cell may, for example, be from a sample isolated from a patient, a related or unrelated haematopoietic transplant donor, a completely unconnected donor, from cord blood, differentiated from an embryonic cell line, differentiated from an inducible progenitor cell line, or derived from a transformed cell line.

In a seventh aspect, the present invention provides a pharmaceutical composition comprising a plurality of cells according to the first aspect of the invention.

In an eighth aspect, the present invention provides a method for treating and/or preventing a disease, which comprises the step of administering a pharmaceutical composition according to the seventh aspect of the invention to a subject.

The method of the eight aspect of the invention may comprise the following steps:
(i) isolation of a cell-containing sample from a subject;
(ii) transduction or transfection of the cells with: a nucleic acid construct of the second aspect of the invention; a first nucleic acid sequence and second nucleic acid sequence as defined in the third aspect of the invention; and/or a first vector and a second vector as defined in the fourth aspect of the invention, or a vector according to the fifth aspect of the invention; and
(iii) administering the cells from (ii) to the subject.

The disease may be cancer. The cancer may be a B cell malignancy.

In a ninth aspect, the present invention relates to a pharmaceutical composition according to the seventh aspect of the invention for use in treating and/or preventing a disease.

In a tenth aspect, the present invention provides the use of a cell according to the first aspect of the invention in the manufacture of a medicament for treating and/or preventing a disease.

The cell of the first aspect of the invention is capable of killing a target cell. The target cell may express one or more antigens recognised by the first and/or second CAR of the cell of the present invention. The target cell may express a high level of one or more inhibitory receptors, such as PDL1. Where a target cell expresses a high level of an inhibitory receptor such as PDL1, recognition by a T-cell would recruit inhibitory phosphatases such as PD1 to the T-cell:target-cell synapse. In these circumstances it is beneficial to increase the amount of kinase (i.e. amplify phosphorylation) in order to allow T-cell activation to occur.

The present inventors have developed a new CAR system which involves two CARs, one with an activating endodomain and one with a phosphorylation amplifying endodomain. When the two CARs recognise their target antigen, the phosphorylation amplifying endodomain causes the equilibrium between phosphorylation:dephosphorylation to tip in favour of phosphorylation, amplifying cell signalling via the activating endodomain. This provides a system for increasing T-cell activation and therefore T-cell proliferation, persistence and engraftment which does not rely on cytokine administration or production.

### DESCRIPTION OF THE FIGURES

FIG. 1: (a) Schematic diagram illustrating a classical CAR. (b) to (d): Different generations and permutations of CAR endodomains: (b) initial designs transmitted ITAM signals alone through FcεR1-γ or CD3-ξ endodomain, while later designs transmitted additional (c) one or (d) two activating endodomains, such as 4-1BB, in the same compound.

FIG. 2: Schematic diagram of a first and second chimeric antigen receptor (CAR) of the invention. The first CAR has an endodomain comprising a phosphorylation amplifying (Pa) endodomain. The second CAR has an endodomain comprising an activating endodomain. The antigen-binding domains of the two chimeric antigen receptors bind different epitopes on the same antigen. For example, binder 1 of the first CAR binds to a first epitope, and binder 2 of the second CAR binds to a second epitope of the same antigen. The spacer may be different, for example, spacer 1 on the first CAR may be longer than spacer 2 of the second CAR. Note: Although only one chain is shown, the CARs in this system may be homodimers.

FIG. 3: Schematic diagram of an alternative first and second chimeric antigen receptor (CAR) of the invention. The first and second CARs have a similar structure to the ones shown in FIG. 2 in terms of endodomains. The difference is that the antigen-binding domains of the two chimeric antigen receptors bind the same epitopes on the antigen. For example binder 1 of the first CAR and the second CAR both bind to epitope 1 of an antigen, and they may comprise identical antigen-binding portions. Similarly, the spacer on the first and second CAR may be the same. Note: Although only one chain is shown, the CARs in this system may be homodimers.

FIG. 4: Schematic diagram of a CAR T cell, which comprises a first CAR and a second CAR, each of which binds to different epitopes on the same antigen. The first CAR comprises an inotuzumab scFv antigen binding domain which is binds to CD22, a CD8 Stalk transmembrane domain and any one or more of the following phosphorylation amplifying endodomains (X) comprising either the tyrosine kinase domain of Fyn, Src, Lck or mutated Lck (Y505F) or the intracellular domain of CD4 or CD8 coreceptors. The second CAR comprises a CD22ALab antigen binding domain also capable of binding to an epitope on a CD22 antigen, a Hinge transmembrane domain and activating endodomain comprising CD3-ξ and 4-1BB.

FIG. 5: Cytotoxicity (72 h) of CAR T cell constructs for Raji target cells. To measure cytotoxic capacity of the panel of constructs comprising a phosphorylating amplifying endodomain, each CAR was challenged against the Raji cell line. 72 hours after the T cells and Raji cells were co-cultured, the absolute number of Raji target cells was calculated, and the number in the CAR-T cell containing sample normalised according to the target number in the non-transduced (NT) condition. The normalised data are expressed as a percentage of cell survival. The construct having a first CAR comprising a phosphorylating amplifying endodomain comprising the tyrosine kinase domain of a Fyn shows the lowest overall percentage of cell survival compared to the other phosphorylating amplification endodomains.

FIG. 6: T-cell proliferation (day 7) following co-culture with Raji cells. CD56-depeleted CAR expressing T cells were analysed by flow cytometry to measure the dilution of the Cell Trace Violet (CTV) which occurs as the T-cells divide. The T cells labelled with CTV are excited with a 405 nm (violet) laser. Proliferation of the CAR T cells comprising a phosphorylating amplifying endodomain is shown to be significantly improved for the donor tested compared to the constructs lacking a phosphorylating amplifying endodomain.

FIG. 7(a): IFN-γ cytokine production (72 h) after co-culture with Raji cells. T-cells expressing the panel of CAR constructs having different phosphorylating amplifying endodomains were compared for IFN-γ secretion after 72 h co-culture with Raji target cells.

FIG. 7(b): IL-2 cytokine production (72 h) after co-culture with Raji cells. T-cells expressing the panel of CAR constructs having different phosphorylating amplifying endodomains were compared for IL-2 secretion after 72 h co-culture with Raji target cells.

FIG. 8—Expression data of BCMA on primary myeloma cells

Myeloma cells from bone marrow samples from 39 multiple myeloma patients were isolated by a CD138+ magnetic bead selection. These cells were stained with the anti-BCMA monoclonal antibody J6MO conjugated with PE (GSK). Antigen copy number was quantified using PE Quantibrite beads (Becton Dickenson) as per the manufacturer's instructions. A box and whiskers plot of antigen copy number is presented along with the range, interquartile and median values plotted. It was found that the range is 348.7-4268.4 BCMA copies per cell with a mean of 1181 and a median of 1084.9.

FIG. 9—Schematic diagram illustrating the distance parameters at a T-cell:target cell synapse.

FIG. 10—Schematic diagram of an alternative first and second chimeric antigen receptor (CAR) of the invention. The first and second CARs have a similar structure to the ones shown in FIG. 3 in terms of endodomains. The difference is that the antigen-binding domains of the two chimeric antigen receptors bind to different target antigens.

FIG. 11—Schematic diagram of an alternative first and second chimeric antigen receptor (CAR) of the invention. As for the arrangement shown in FIG. 10, the antigen-binding domains of the first and second CARs bind to different target antigens. In this arrangement, one CAR comprises a phosphorylation amplifying domain together with a co-stimulatory domain.

FIG. 12—Cytotoxicity (72 h) of CAR T cells expressing various CAR systems against SupT1 target cells expressing the target antigen CD22 at an average copy number of 0, 255, 634, 1090 or 78,916 copies per cell

FIG. 13—T-cell proliferation (day 7) following co-culture with SupT1 target cells expressing the target antigen CD22 at an average copy number of 441 or 78,916 copies per cell.

### DETAILED DESCRIPTION

Chimeric Antigen Receptors (Cars)

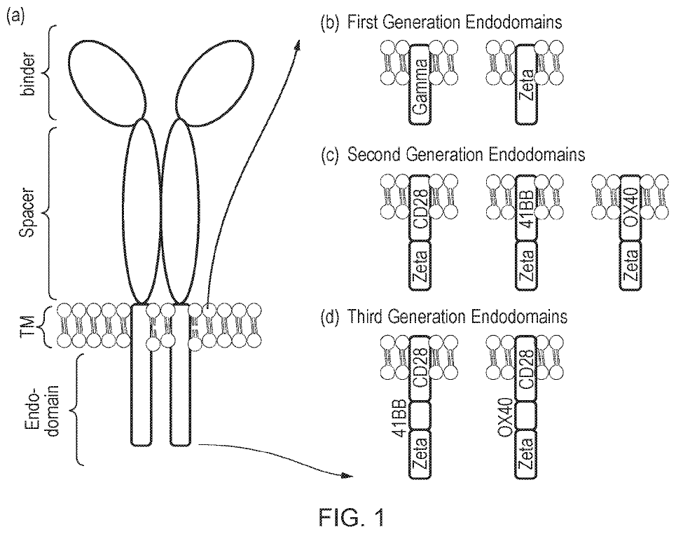

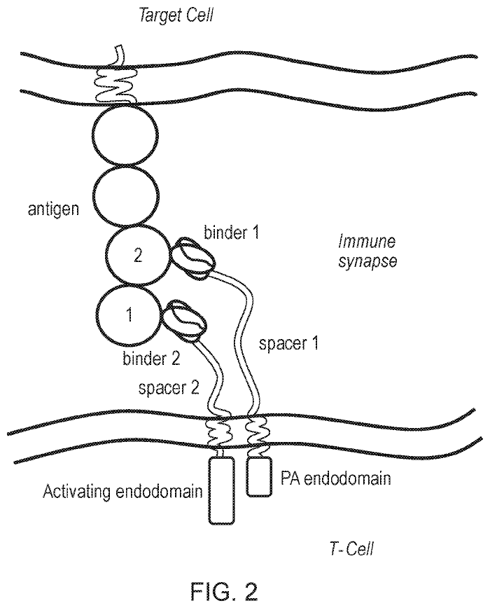

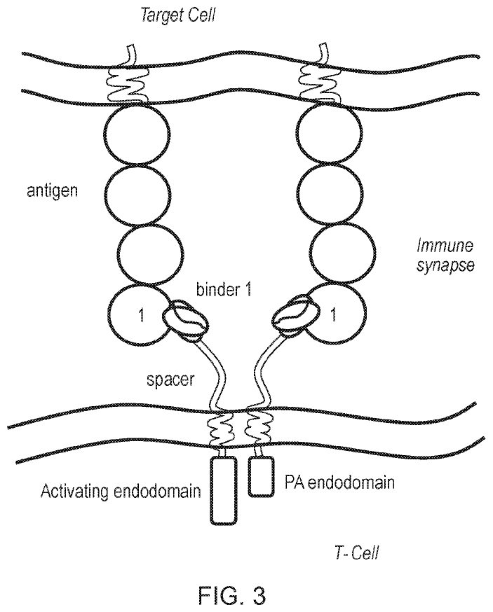

CARs, which are shown schematically in FIG. 1, are chimeric type I trans-membrane proteins which connect an extracellular antigen binding domain (binder) to an endodomain. The binder is typically a single-chain variable fragment (scFv) derived from a monoclonal antibody (mAb) such as inotuzumab, but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the different antigens, and/or indeed the different epitopes of the same antigen. A transmembrane domain anchors the protein in the cell membrane and connects the spacer to the endodomain. In a classical, activating CAR, the endodomain comprises an intracellular signalling domain.

Early CAR designs had endodomains derived from the intracellular parts of either the γ chain of the FcεR1 or CD3-ξ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3-ξ results in second generation receptors which can transmit an activating and co-stimulatory signal simultaneously after antigen recognition. The co-stimulatory domain commonly used is that of CD28. This supplies a potent co-stimulatory signal—namely immunological signal 2 (IL2), which triggers T-cell proliferation. Receptors described herein include TNF receptor family endodomains, such as the closely related OX40 and 4-1BB, which transmit survival signals, as shown in FIG. 1(c). Even more potent third generation CARs have also been described which have endodomains capable of transmitting activation, proliferation and survival signals, as shown in FIG. 1(d). Receptors in the present invention comprise activating endodomains that either include a 4-1BB or do not include a 4-1BB domain.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus, the CAR is capable of directing specificity and cytotoxicity of the T cell towards tumour cells expressing the targeted antigen.

T-Cell Activation (Resting State):

T-cell receptors (TCR) and CARs cause T-cell signalling by stimulating tyrosine phosphorylation: the addition of a phosphate group to the amino acid tyrosine on a protein. Tyrosine-protein kinases transfer a phosphate group from ATP to a tyrosine residue in a protein. These enzymes can be divided into two main groups: receptor tyrosine kinases (RTK), which are transmembrane proteins involved in signal transduction; and cytoplasmic/non-receptor tyrosine kinases, which act as regulatory proteins, playing key roles in cell differentiation, motility, proliferation, and survival.

In the resting state, the molecules involved in T cell-signalling are repeatedly colliding by means of diffusion. For example, the TCR/CD3 complex is constantly being phosphorylated by a Src-family kinase (SFK), a tyrosine kinase, either free or non-covalently bound to the cytoplasmic tails of CD4 or CD8 co-receptors.

In turn, the complex is continuously dephosphorylated by tyrosine phosphatases, such as CD45 and CD148. The continuous phosphorylation and dephosphorylation happens in a random manner, and in the absence of ligand binding, this equilibrium favours dephosphorylation and the overall phosphorylation of TCR is low such that T-cell activation does not proceed.

T Cell Activation (Activated State):

Antigen recognition by the TCR is the first step in T-cell activation. At the start of the signalling cascade, the immunoreceptor tyrosine-based activation motifs (ITAMs) of CD3 are phosphorylated by a SFK (Lck) and then bound by another kinase called ZAP70. After ZAP70 binds to CD3, co-receptors CD4 or CD8 become associated with the TCR/CD3 complex and bind to the major compatibility complex (MHC). CD8 co-receptor association with the complex stabilises the TCR-MHC peptide (MHCp) interaction and the recruited/free Lck continues the phosphorylation of CD3 elements, ZAP70, as well as many other downstream targets.

The TCR-MHCp and CAR-target cell antigen complex spans a short length. This forms small zones of close contact, from which the inhibitory CD45 and CD148 phosphatase molecules with ectodomains too large to fit are excluded. CD45 steric exclusion extends the phosphorylation half-lives of TCR/MHCp complexes or CAR-target cell antigen complexes, which are trapped within the close-contact zone. In the absence of CD45, Lck is favoured, which phosphorylates ITAMs. Such prolonged phosphorylation allows more time for ZAP-70 recruitment, its activation by phosphorylation, and subsequent phosphorylation of adaptor proteins. This extended and increased level of phosphorylation tips the balance of the resting T cell phosphorylation/dephosphorylation equilibrium state towards phosphorylation, meeting the T cell activation threshold required for activation to proceed.

A central mechanism of CAR T-cell activation is therefore the equilibrium shift of phosphorylation/dephosphorylation events that occur upon target cell antigen recognition.

The present inventors have devised a CAR system which effectively lowers the T cell activation threshold (of phosphorylation events required for T cell activation to proceed) by manipulating the equilibrium towards phosphorylation. This is done by including a phosphorylation amplification endodomain into the CAR system.

In one aspect, the present invention relates to a cell which co-expresses a first CAR and a second CAR, wherein the first CAR comprises a phosphorylation amplifying endodomain and wherein the second CAR comprises an activating endodomain.

Phosphorylation Amplifying (PA) Endodomain

The phosphorylating amplifying endodomain of the first CAR of the cell of the present invention is an intracellular domain which can either directly or indirectly amplify the phosphorylation of an ITAM. For example, the PA endodomain may be able to directly or indirectly amplify the phosphorylation of one or more ITAM(s) present in the activating endodomain of the second CAR.

An example of direct amplification of phosphorylation is phosphorylation by a kinase. The tyrosine kinase domain of a SRK protein can directly phosphorylate the tyrosine residues on ITAMs.

An example of indirect amplification of phosphorylation is via recruitment of a kinase. For example, the intracellular domains of CD4 and CD8 coreceptors indirectly indirectly amplify phosphorylation of ITAMs by clustering with the CAR T cell antigen complex, recruiting further SRK proteins, which then directly phosphorylate the ITAMs. These coreceptors also further stabilise the CAR T cell antigen complex, which optimizes signalling through the CAR T cell receptor complex required for T cell activation.

The phosphorylating amplifying domain of the first CAR may comprise all or part of the tyrosine kinase domain of an SRK protein.

Alternatively or additionally, the phosphorylating amplifying domain of the first CAR may comprise all or part of the intracellular domain of a CD4 coreceptor or the CD8 coreceptor.

SRC Family Kinase (SRK)

SRK member proteins contain a 14-carbon myristic acid moiety attached to an SH4 domain, a SH3 domain followed by an SH2 domain, an SH2-kinase linker, a tyrosine kinase domain (also known as a catalytic domain or a SH1 domain), and a C-terminal regulatory segment. The tyrosine kinase domain of a SRK protein is an example of a phosphorylating amplifying endodomain in the context of the present invention.

SRK is a family of non-receptor tyrosine kinases that includes nine members: Src, Yes, Fyn and Frg, forming the SrcA subfamily, Lck, Hck, Blk and Lyn in the SrcB subfamily and Frk in its own subfamily. The SrcA and SrcB subfamilies are specific to vertebrates; however, Src homologs exist in organisms as diverse as unicellular choanoflagellates.

SRKs interact with many cellular cytosolic, nuclear and membrane proteins, modifying these proteins by phosphorylation of tyrosine residues. The SH3, SH2 and kinase domains of SFK display large sequence and structural similarity. They also have in common a myristoylated and/or palmitoylated membrane anchoring region in the N-terminus, including positively charged residues (Arg and/or Lys), known as the SH4 domain. The activity of SFKs is largely controlled by the equilibrium between phosphorylation and dephosphorylation at a C-terminal inhibitory tyrosine and an activating tyrosine in the catalytic domain.

The phosphorylating amplifying domain of the first CAR of the cell may comprise the tyrosine kinase domain of a SRK protein. Sequences of such domains are disclosed as SEQ ID NOs: 1, 3, 5, and 7.

Alternatively, the phosphorylating amplifying domain of the first CAR of the cell may comprise the full-length sequence of a SRK protein. Sequences of such domains are disclosed below as SEQ ID NO: 2, 4, 6 and 8.

The PA domain of the first CAR may comprise a variant of one of the sequence showns as SEQ ID No. 1 to 8 having at least 80, 85, 90, 95, 98 or 99% sequence identity, provided that the variant sequence retains the capacity to amplify phosphorylation. The variant should retain tyrosine kinase activity.

FYN

Fyn (UniProt ID: P06241) is a 59 kda member of the SRK family, typically associated with T cell and neuronal signalling in development and normal cell physiology. It encodes a membrane-associated non-receptor tyrosine kinase that plays a role in many biological processes including regulation of cell growth and survival, cell adhesion, integrin-mediated signaling, cytoskeletal remodelling, cell motility, immune response and axon guidance. Fyn is phosphorylated on its C-terminal tail within the catalytic domain (also known as the tyrosine kinase domain). Fyn participates in the downstream signaling pathways that lead to T-cell differentiation and proliferation following T-cell receptor (TCR) stimulation. Fyn also participates in negative feedback regulation of TCR signaling through phosphorylation.

The inventors have discovered that Fyn is a particularly effective phosphorylating amplification domain for use in a cell comprising a first CAR and a second CAR of the present invention. For example, the Fyn-CAR construct shown in FIG. 5 has the lowest overall percentage of cell survival compared to the other phosphorylating amplification domains.

Tyrosine kinase domain Fyn (SEQ ID NO: 1)
LQLIKRLGNGQFGEVWMGTWNGNTKVAIKTLKPGTMSPESFLEEAQIMKK
LKHDKLVQLYAVVSEEPIYIVTEYMNKGSLLDFLKDGEGRALKLPNLVDM
AAQVAAGMAYIERMNYIHRDLRSANILVGNGLICKIADFGLARLIEDNEY
TARQGAKFPIKWTAPERALYGRFTIKSDVWSFGILLTELVTKGRVPYPGM
NNREVLEQVERGYRMPCPQDCPISLHELMIHCWKKDPEERPTFEYLQSFL
EDYF
Full-length Fyn (SEQ ID NO: 2):
GCVQCKDKEATKLTEERDGSLNQSSGYRYGTDPTPQHYPSFGVTSIPNYN
NFHAAGGQGLTVFGGVNSSSHTGTLRTRGGTGVTLFVALYDYEARTEDDL
SFHKGEKFQILNSSEGDWWEARSLTTGETGYIPSNYVAPVDSIQAEEWYF
GKLGRKDAERQLLSFGNPRGTFLIRESETTKGSYSLSIRDWDDMKGDHVK
HYKIRKLDNGGYYITTRAQFETLQQLVQHYSERAAGLCCRLVVPCHKGMP
RLTDLSVKTKDVWEIPRESLQLIKRLGNGQFGEVWMGTWNGNTKVAIKTL
KPGTMSPESFLEEAQIMKKLKHDKLVQLYAVVSEEPIYIVTEYMNKGSLL
DFLKDGEGRALKLPNLVDMAAQVAAGMAYIERMNYIHRDLRSANILVGNG
LICKIADFGLARLIEDNEYTARQGAKFPIKWTAPERALYGRFTIKSDVWS
FGILLTELVTKGRVPYPGMNNREVLEQVERGYRMPCPQDCPISLHELMIH
CWKKDPEERPTFEYLQSFLEDYFTATEPQYQPGENL

Src

Src (UniProt ID: P12931) is a 59 kDa member of the SRK family. This proto-oncogene also known as c-Src, is a non-receptor tyrosine kinase protein. Src is activated following engagement of different classes of cellular receptors including immune response receptors, integrins and other adhesion receptors, receptor protein tyrosine kinases, G protein-coupled receptors as well as cytokine receptors. Src participates in signaling pathways that control a diverse spectrum of biological activities including gene transcription, immune response, cell adhesion, cell cycle progression, apoptosis, migration, and transformation.

Tyrosine kinase domain of Src (SEQ ID NO: 3)
LRLEVKLGQGCFGEVWMGTWNGTTRVAIKTLKPGTMSPEAFLQEAQVMKK
LRHEKLVQLYAVVSEEPIYIVTEYMSKGSLLDFLKGETGKYLRLPQLVDM
AAQIASGMAYVERMNYVHRDLRAANILVGENLVCKVADFGLARLIEDNEY
TARQGAKFPIKWTAPEAALYGRFTIKSDVWSFGILLTELTTKGRVPYPGM
VNREVLDQVERGYRMPCPPECPESLHDLMCQCWRKEPEERPTFEYLQAFL
EDYF
Full-length SRc (SEQ ID NO: 4):
MGSNKSKPKDASQRRRSLEPAENVHGAGGGAFPASQTPSKPASADGHRGP
SAAFAPAAAEPKLFGGFNSSDTVTSPQRAGPLAGGVTTFVALYDYESRTE
TDLSFKKGERLQIVNNTEGDWWLAHSLSTGQTGYIPSNYVAPSDSIQAEE
WYFGKITRRESERLLLNAENPRGTFLVRESETTKGAYCLSVSDFDNAKGL
NVKHYKIRKLDSGGFYITSRTQFNSLQQLVAYYSKHADGLCHRLTTVCPT
SKPQTQGLAKDAWEIPRESLRLEVKLGQGCFGEVWMGTWNGTTRVAIKTL
KPGTMSPEAFLQEAQVMKKLRHEKLVQLYAVVSEEPIYIVTEYMSKGSLL
DFLKGETGKYLRLPQLVDMAAQIASGMAYVERMNYVHRDLRAANILVGEN
LVCKVADFGLARLIEDNEYTARQGAKFPIKWTAPEAALYGRFTIKSDVWS
FGILLTELTTKGRVPYPGMVNREVLDQVERGYRMPCPPECPESLHDLMCQ
CWRKEPEERPTFEYLQAFLEDYFTSTEPQYQPGENL

Lck

Lck (UniPort ID: P06239) is a 56 kDa on-receptor tyrosine-protein kinase that plays an essential role in the selection and maturation of developing T-cells in the thymus and in the function of mature T-cells. It also plays a key role in T-cell antigen receptor (TCR)-linked signal transduction pathways. Lck is constitutively associated with the cytoplasmic portions of the CD4 and CD8 surface receptors, and association of the TCR with a peptide antigen-bound MHC complex facilitates the interaction of CD4 and CD8 with MHC class II and class I molecules, respectively, thereby recruiting the associated Lck protein to the vicinity of the TCR/CD3 complex. Lck then phosphorylates tyrosines residues within the immunoreceptor tyrosine-based activation motifs (ITAM) of the cytoplasmic tails of the TCR-gamma chains and CD3 subunits, initiating the TCR/CD3 signaling pathway. Once stimulated, the TCR recruits the tyrosine kinase ZAP70, that becomes phosphorylated and activated by Lck. Following this, a large number of signaling molecules are recruited, ultimately leading to lymphokine production. Lck also contributes to signaling by other receptor molecules.

Tyrosine kinase domain of Lck (SEQ ID NO: 5):
LKLVERLGAGQFGEVWMGYYNGHTKVAVKSLKQGSMSPDAFLAEANLMKQ
LQHQRLVRLYAVVTQEPIYIITEYMENGSLVDFLKTPSGIKLTINKLLDM
AAQIAEGMAFIEERNYIHRDLRAANILVSDTLSCKIADFGLARLIEDNEY
TAREGAKFPIKVVTAPEAINYGTFTIKSDVWSFGILLTEIVTHGRIPYPG
MTNPEVIQNLERGYRMVRPDNCPEELYQLMRLCWKERPEDRPTFDYLRSV
LEDFF
Full-length Lck (SEQ ID NO: 6):
MGCGCSSHPEDDWMENIDVCENCHYPIVPLDGKGTLLIRNGSEVRDPLVT
YEGSNPPASPLQDNLVIALHSYEPSHDGDLGFEKGEQLRILEQSGEVWVK
AQSLTTGQEGFIPFNFVAKANSLEPEPWFFKNLSRKDAERQLLAPGNTHG
SFLIRESESTAGSFSLSVRDFDQNQGEVVKHYKIRNLDNGGFYISPRITF
PGLHELVRHYTNASDGLCTRLSRPCQTQKPQKPWWEDEWEVPRETLKLVE
RLGAGQFGEVWMGYYNGHTKVAVKSLKQGSMSPDAFLAEANLMKQLQHQR
LVRLYAVVTQEPIYIITEYMENGSLVDFLKTPSGIKLTINKLLDMAAQIA
EGMAFIEERNYIHRDLRAANILVSDTLSCKIADFGLARLIEDNEYTAREG
AKFPIKVVTAPEAINYGTFTIKSDVWSFGILLTEIVTHGRIPYPGMTNPE
VIQNLERGYRMVRPDNCPEELYQLMRLCWKERPEDRPTFDYLRSVLEDFF
TATEGQYQPQP

The Lck mutant (Y505F) provided herein is a constitutively active variant showing significantly increased cytotoxicity (see FIGS. 5, 6 and 7a and 7b). The Y505F mutant protects against CSK phosphorylation at the 505 tyrosine residue.

Tyrosine kinase domain of Lck_Y505F (SEQ ID NO: 7)
LKLVERLGAGQFGEVWMGYYNGHTKVAVRSLKQGSMSPDAFLAEANLMKQ
LQHQRLVRLYAVVTQEPIYIITEYMENGSLVDFLKTPSGIKLTINKLLDM
AAQIAEGMAFIEERNYIHRDLRAANILVSDTLSCKIADFGLARLIEDNEY
TAREGAKFPIKWTAPEAINYGTFTIKSDVWSFGILLTEIVTHGRIPYPGM
TNPEVIQNLERGYRMVRPDNCPEELYQLMRLCWKERPEDRPTFDYLRSVL
EDFF
Full length Lck_Y505F (SEQ ID NO: 8):
MGCGCSSHPEDDWMENIDVCENCHYPIVPLDGKGTLLIRNGSEVRDPLVT
YEGSNPPASPLQDNLVIALHSYEPSHDGDLGFEKGEQLRILEQSGEWWWK
AQSLTTGQEGFIPFNFVAKANSLEPEPWFFKNLSRKDAERQLLAPGNTHG
SFLIRESESTAGSFSLSVRDFDQNQGEVVKHYKIRNLDNGGFYISPRITF
PGLHELVRHYTNASDGLCTRLSRPCQTQKPQKPWWEDEWEVPRETLKLVE
RLGAGQFGEVWMGYYNGHTKVAVRSLKQGSMSPDAFLAEANLMKQLQHQR
LVRLYAVVTQEPIYIITEYMENGSLVDFLKTPSGIKLTINKLLDMAAQIA
EGMAFIEERNYIHRDLRAANILVSDTLSCKIADFGLARLIEDNEYTAREG
AKFPIKWTAPEAINYGTFTIKSDVWSFGILLTEIVTHGRIPYPGMTNPEV
IQNLERGYRMVRPDNCPEELYQLMRLOWKERPEDRPTFDYLRSVLEDFFT
ATEGQFQPQP

Intracellular CD4 and CD8 Co-Receptors

Membrane proteins CD4 and CD8 co-receptors are expressed on T helper (Th) cells and cytotoxic T lymphocytes (CTL). They are non-polymorphic cell surface glycoproteins expressed on subsets of thymocytes and mature peripheral T cells. Generally, CD4 is expressed on Th cells that recognise antigens in association with class II MHC molecules, and CD8 is expressed on CTLs that recognize antigens in association with class I MHC molecules. CD4 and CD8 actively participate as co-receptors during T cell signalling and enhance antigen responsiveness mediated by TCR.

It has been shown that CD4 and CD8 associated with equal amounts of Lck, both enhanced IL2 production equivalently when cross-linked with suboptimal levels of anti-TCR antibody. The cytoplasmic tail portion of CD8 and CD4 coreceptors interacts with the cytoplasmic tail of CD3-ξ.

The phosphorylating amplifying domain of the first CAR of the cell may comprise the intracellular domain (also known as the cytoplasmic tail) of a CD4 or CD8 coreceptor. Sequences of such domains are disclosed as SEQ ID NOs: 9 and 11, respectively.

Alternatively, the phosphorylating amplifying domain of the first CAR of the cell may comprise the full-length sequence of a CD4 or CD8 coreceptor, or truncations thereof. The full-length sequence of the CD4 and CD8 coreceptor are provided below as SEQ ID Nos: 10 and 12, respectively.

The PA domain of the first CAR may comprise a variant of one of the sequence shown as SEQ ID No. 9 to 12 having at least 80, 85, 90, 95, 98 or 99% sequence identity, provided that the variant sequence retains the capacity to amplify phosphorylation. The variant should retain the capacity to bind an SRK kinase such as Lck.

CD4 Coreceptor

CD4 (UniProt ID: P01730) is a co-receptor that assists the T cell receptor (TCR) in communicating with an antigen-presenting cell. Using its intracellular domain, CD4 amplifies the signal generated by the TCR by recruiting Lck, which is essential for activating many molecular components of the signaling cascade of an activated T cell Various types of Th cells are thereby produced. CD4 also interacts directly with MHC class II molecules on the surface of the antigen-presenting cell using its extracellular domain. The extracellular domain adopts an immunoglobulin-like beta-sandwich with seven strands in 2 beta sheets.

During antigen presentation, both the TCR complex and CD4 are recruited to bind to different regions of the MHCII molecule (α1/β1 and β2, respectively). Close proximity between the TCR complex and CD4 in this situation means the Lck kinase bound to the cytoplasmic tail of CD4 is able to tyrosine-phosphorylate the Immunoreceptor tyrosine activation motifs (ITAM) present on the cytoplasmic domains of CD3. Phosphorylated ITAM motifs on CD3 recruits and activates SH2 domain-containing protein tyrosine kinases (PTK) such as ZAP70 to further mediate downstream signal transduction via tyrosine phosphorylation, leading to transcription factor activation including NF-κB and consequent T cell activation.

Cytoplasmic tail of CD4 (SEQ ID NO: 9)
CVRCRHRRRQAERMSQIKRLLSEKKTCQCPHRFQKTCSPI
Full-length CD4 (SEQ ID NO: 10):
MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVQPMALI
VLGGVAGLLLFIGLGIFFCVRCRHRRRQAERMSQIKRLLSEKKTCQCPHR
FQKTCSPI

CD8 Coreceptor

CD8 (UniProt ID: P01732) plays a key role in signal transduction by recruiting essential signaling components to the cytoplasmic side of the TCR-CD3-ξ complex. Evidence by Wooldridge et al., (2005) J Biol Chem; 280(30):27491-501 shows that although CD8 and TCR do not bind cooperatively to pMHCI, TCR associates with CD8 on the T cell surface and the interaction stabilises the T cell receptor antigen complex.

Cytoplasmic tail of CD8 (SEQ ID NO: 11)
LYCNHRNRRRVCKCPRPVVKSGDKPSLSARYV
Full-length CD8 (SEQ ID NO: 12):
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV
CO-STIMULATORY ENDODOMAIN

The first CAR may comprise one or more co-stimulatory domains in addition to the PA domain. The co-stimulatory domain(s) may be membrane proximal or membrane-distal in comparison with the PA domain.

The costimulatory endodomain may comprises one or more of the following: CD28, OX40 and 4-1BB endodomain, which are described in more detail below.

The costimulatory domain may comprise a CD28 endodomain in combination with an OX40 or 41BB endodomain.

Antigen Binding Domain

The antigen-binding domain is the portion of the CAR which recognizes the antigen. Numerous antigen-binding domains are known in the art, including those based on the antigen-binding site of an antibody, antibody mimetics, and T-cell receptors. For example, the antigen-binding domain may comprise: a single-chain variable fragment (ScFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

A ‘target antigen’ is an entity, which is specifically recognised and bound by the antigen-binding domain of a CAR. The term ‘ligand’ is used synonymously with ‘antigen’. An ‘epitope’ refers to the specific binding site to which the antigen-binding domain (e.g., scFv portion of the CAR) binds.

In the context of the first aspect of the invention, the antigen-binding domain of first CAR and second CAR of the cell may recognise different antigens. Alternatively, the antigen-binding domain of the first CAR and the second CAR of the cell may recognise the same antigen. In one embodiment, the antigen binding domains of the first CAR and second CAR may recognise distinct epitopes of the same antigen. Where antigen binding domains of the first CAR and second CAR recognise distinct epitopes of the same antigen, it is possible for the first CAR and second CAR to bind the same antigen molecule which automatically brings the PA domain of the first CAR into proximity with the activating endodomain of the second CAR.

In the CAR system of the present invention, maximal activation is achieved when both the first and second CAR bind their target antigen. Where the first CAR and second CAR bind different antigens, this provides an extra level of safety, as maximal activation will only occur when two targets e.g. two tumour-specific antigens are present. This is useful in the field of oncology as indicated by the Goldie-Coldman hypothesis: sole targeting of a single antigen may result in tumour escape by modulation of said antigen due to the high mutation rate inherent in most cancers. By simultaneously targeting two different antigens, the probability of such escape is reduced.

The target antigen may be an antigen present on a cancer cell, for example a tumour-associated antigen. It may, for example, be one of the antigens listed in Table 1.

Target Antigen Density

The target antigen for the first and/or second CAR may be expressed at relatively low density on the target cell. The target antigen for the first and/or second CAR may be of a size or configuration that causes it to be excluded from a T-cell:target cell synapse.

The target antigen may be expressed at a low density on the cell surface. Thus the cells of the present invention may be capable of killing target cells, such as cancer cells, which express a low density of the TAA. Examples of TAAs which are known to be expressed at low densities in certain cancers include, but are not limited to, ROR1 in CLL, Typr-1 in melanoma, BCMA and TACI in myeloma, CD22 in B-cell malignancies and ALK in Neuroblastoma.

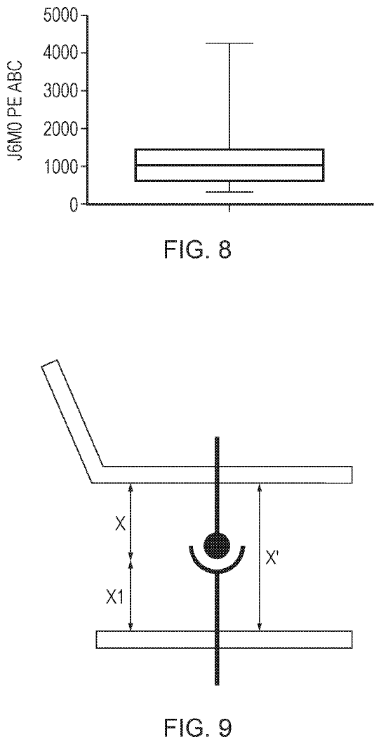

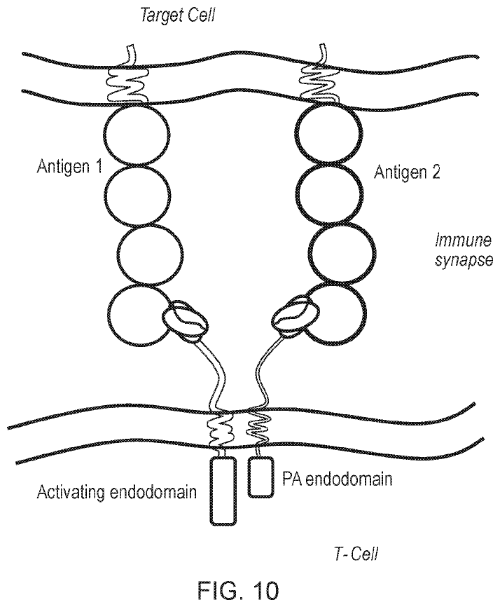

Example 4 describes a study investigating the expression of BCMA on myeloma cells. It was found that the range of BCMA copy number on a myeloma cell surface is low: at 348.7-4268.4 BCMA copies per cell with a mean of 1181 and a median of 1084.9 (FIG. 8).

Example 5 demonstrates that the inclusion of a CAR expressing a Pa domain increases killing and T-cell persistence with target cells expressing low densities of the target antigen CD22. The effect was observed for target cells expressing approximately 1090, 634, 441 and 255 copies of target antigen per cell on average.

The mean copy number of the target antigen, for example the target antigen for the second CAR, may be fewer than about 10,000; 5,000; 3,000; 2,000; 1,000; 500 copies or 250 per target cell.

The copy number of an antigen on a cell, such as a cancer cell may be measured using standard techniques, such as using PE Quantibrite beads as described in Example 4.

The copy number of the target antigen for the first CAR may be different from the copy number of the target antigen for the second CAR.

For example, the target antigen for the second CAR (which comprises an activating endodomain) may be expressed by the target cell at a lower antigen density that the target antigen for the first CAR (which comprises a phosphorylation amplifying endodomain).

The target antigen for the second CAR (which comprises an activating endodomain) may be expressed by the target cell at an average copy number of 1000, 500 or 250 copies per cell or fewer. The target antigen for the first CAR (which comprises a phosphorylation amplifying endodomain) may be expressed by the target cell at an average copy number of at least 1000; 2,000; 3,000; 5,000 or 10,000 copies per cell.

The following table summarises examples of high density tissue-specific antigens and low-density tumour-specific target antigens for various diseases. The high density tissue specific antigens are suitable for targeting wih the first CAR which comprises a PA domain and optionally a co-stimulatory domain. The low density tumour specific antigens are suitable for targeting with the second CAR which comprises an activating endodomain.

| | | Tumour specific |
|---|---|---|
| Disease | Tissue specific antigen | low density antigen |
| Multiple Myeloma | CD38, CD56, CD138 | BCMA |
| B-cell Acute Lymphoblastic | CD10 | CD22 |
| Leukaemia | | |
| Chronic Lymphocytic | ROR1 | CD19 |
| Leukaemia | | |
| Neuroblastoma | | ALK |
| T-cell acute Lymphoblastic | CD2, CD5, CD7 | CD21 |
| Leukaema | | |

The second CAR may bind to one of the following target antigens: B cell maturation antigen (BCMA), transmembrane activator and calcium modulator and cyclophilin ligand interactor (TACI), CD22, CD19, CD21 and Anaplastic Lymphoma Kinase (ALK).

The target antigen for the second CAR may be BCMA and/or TACI and the target antigen for the first CAR may be CD38, CD56 or CD138.

The target antigen for the second CAR may be CD22 and the target antigen for the first CAR may be CD10.

The target antigen for the second CAR may be CD19 and the target antigen for the first CAR may be ROR1.

The target antigen for the second CAR may be CD21 and the target antigen for the first CAR may be CD2, CD5 or CD7.

An antigen binding domain against CD38 may be derived from daratumumab. A suitable daratumumab scFv sequence is shown as SEQ ID No. 13 below.

SEQ ID No. 13 - Daratumumab scFv
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIKRSGGGGSGGGGSGGGGSEVQLLESGGGLVQPGGSLRLSCAVSG
FTFNSFAMSWVRQAPGKGLEWVSAISGSGGGTYYADSVKGRFTISRDNSK
NTLYLQMNSLRAEDTAVYFCAKDKILWFGEPVFDYWGQGTLVTVSS

SEQ ID No. 14 shows an anti-CD10 scFv sequence.

SEQ ID No. 14 - anti-CD10 scFv
DIVMTQSPDSLAVSLGERATINCSVSSSISSSNLHWYQQKPGQPPKLLIY
GTSNLASGVPDRFSGSGSGTDFTLTISSLQAEDVAVYYCQQWSSYPLTFG
QGTKVEIKRSGGGGSGGGGSGGGGSEVQLVESGGGVVQPGRSLRLSCAAS
GFTFSSFGMHWVRQAPGKGLEWVAYISGGSYTIYYADTVKGRFTISRDNS
KNTLYLQMNSLRAEDTAVYYCARSYGNFWYFDVWGQGTTVTVSS

An antigen binding domain based on the ligand APRIL is capable of binding TACI and BCMA. A truncated APRIL sequence is shown as SEQ ID No. 15. Alternatively an anti-BCMA or anti-TACI scFv may be used. Suitable sequences are shown as SEQ ID No. 16 and 17 respectively.

truncated APRIL
SEQ ID No. 15
SVLHLVPINATSKDDSDVTEVMWQPALRRGRGLQAQGYGVRIQDAGVYLL
YSQVLFQDVTFTMGQVVSREGQGRQETLFRCIRSMPSHPDRAYNSCYSAG
VFHLHQGDILSVIIPRARAKLNLSPHGTFLGFVKL
anti-BCMA scFv
SEQ ID No. 16
DIVLTQSPPSLAMSLGKRATISCRASESVTILGSHLIHWYQQKPGQPPTL
LIQLASNVQTGVPARFSGSGSRTDFTLTIDPVEEDDVAVYYCLQSRTIPR
TFGGGTKLEIKGSTSGSGKPGSGEGSTKGQIQLVQSGPELKKPGETVKIS
CKASGYTFTDYSINWVKRAPGKGLKWMGWINTETREPAYAYDFRGRFAFS
LETSASTAYLQINNLKYEDTATYFCALDYSYAMDYWGQGTSVTVSS
anti-TACI scFv
SEQ ID No. 17
DIVMTQSQKFMSTTVGDRVSITCKASQNVGTAVAWYQQKPGQSPKLLIYS
ASNRYTGVPDRFTGSGSGTDFTLTISNMQSEDLADYFCQQYSSYRTFGGG
TKLEIKRSGGGGSGGGGSGGGGSQVTLKESGPGMLQPSQTLSLTCSFSGF
SLSTFGMGVGWIRQPSGKGLEWLAHIVWVDDAQYSNPALRSRLTISKDTS
KNQVFLKIANVDTADTATYYCSRIHSYYSYDEGFAYWGQGTLVTVSS

SEQ ID No. 18 shows an anti-CD22 scFv sequence suitable for use as an antigen-binding domain.

anti-CD22 scFv
SEQ ID No. 18
DVQVTQSPSSLSASVGDRVTITCRSSQSLANSYGNTFLSWYLHKPGKAPQ
LLIYGISNRFSGVPDRFSGSGSGTDFTLTISSLQPEDFATYYCLQGTHQP
YTFGQGTKVEIKRSGGGGSGGGGSGGGGSEVQLVQSGAEVKKPGASVKVS
CKASGYRFTNYWIHWVRQAPGQGLEWIGGINPGNNYATYRRKFQGRVTMT
ADTSTSTVYMELSSLRSEDTAVYYCTREGYGNYGAWFAYWGQGTLVTVSS

Further anti-CD22 sequences for use as antigen-binding domains are shown below.

Antigen Size/Configuration

The target antigen may comprise a long and/or bulky extracellular domain. Antigens with long and/or bulky extracellular domains are difficult to target with CAR T cells because they may inhibit a close-contact zone forming at the synapse, and permit entry of phosphorylase molecules to dephosphorylate key molecules in T cell signalling, thus inhibiting T cell activation.

Examples of antigens with long and/or bulky extracellular domains include MUC-1, MUC-16, CEACAM-15, CD21 and CD22 (see next section).

All of these antigens are tumour-associated antigens (TAAs). In this respect, MUC-1 is associated with multiple myeloma, ovarian cancer, colon cancer and prostate cancer; MUC-16 is associated with ovarian cancer; CEACAM-15 is associated with colon cancer; and CD22 is associated with lymphoid and myeloid hematological malignancies.

The relative distances at a T-cell:target cell synapse are illustrated schematically in FIG. 9, in which:

X is the length of the extracellular domain of the antigen;

X1 is the length of the CAR; and

X′ is the combined length of X and X1.

The ideal length for X′ for CAR T-cell signalling is about 15 nm.

A “long” antigen in the context of the present invention may be one having an extracellular domain which is greater than about 15, 20, 25 or 30 nm in length.

CD22

In one embodiment of the invention, the first CAR and/or the second CAR bind to the antigen CD22. CD22 is a sugar binding transmembrane protein belonging to the SIGLEC family of lectins. It is found on the surface of mature B cells and on some immature B cells. CD22 is a regulatory molecule that prevents the overactivation of the immune system and the development of autoimmune diseases. The presence of Ig domains makes CD22 a member of the immunoglobulin superfamily. CD22 functions as an inhibitory receptor for B cell receptor (BCR) signaling.

Like CD19, CD22 is widely considered to be a pan-B antigen, although expression on some non-lymphoid tissue has been described. Targeting of CD22 with therapeutic monoclonal antibodies and immunoconjugates has entered clinical testing.

Examples of anti-CD22 CARs are described by Haso et al. (Blood; 2013; 121(7)). Specifically, anti-CD22 CARs with antigen-binding domains derived from m971, HA22 and BL22 scFvs are described. The first and/or second CAR of the cell of the present invention may comprise a CD22 binding domain based on one of these scFvs

CD22 has seven extracellular IgG-like domains, which are commonly identified as Ig domain 1 to Ig domain 7, with Ig domain 7 being most proximal to the B cell membrane and Ig domain 7 being the most distal from the Ig cell membrane.

The positions of the Ig domains in terms of the amino acid sequence of CD22 (http://www.uniprot.org/uniprot/P20273) are summarised in the following table:

| Ig domain | Amino acids |
|---|---|
| 7 | 20-138 |
| 6 | 143-235 |
| 5 | 242-326 |
| 4 | 331-416 |
| 3 | 419-500 |
| 2 | 505-582 |
| 1 | 593-676 |

Where the first and/or second CAR of the present invention bind CD22, it may bind to an epitope on a membrane-proximal Ig domain of CD22, such as Ig domain 5, 6 or 7. The anti-CD22 antibodies HA22 and BL22 (Haso et al 2013 as above), bind to an epitope on Ig domain 5 of CD22.

Alternatively it may bind to an epitope on a membrane-distal Ig domain of CD22, such as Ig domain 1, 2 or 3.

CD22-Antigen Binding Domain: Derived from Inotuzumab (INO)

Where the first and/or second bind CD22, the antigen binding domain which may be derived from Inotuzumab, which has a heavy chain variable region (VH) having complementarity determining regions (CDRs) with the following sequences:

| (SEQ ID No. 19) | |
|---|---|
| CDR1 | NYWIH; |
| (SEQ ID No. 20) | |
| CDR2 | GINPGNNYATYRRKFQG |
| (SEQ ID No. 21) | |
| CDR3 | EGYGNYGAWFAY; |

a light chain variable region (VL) having CDRs with the following sequences:

| (SEQ ID No. 22) | |
|---|---|
| CDR1 | RSSQSLANSYGNTFLS; |
| (SEQ ID No. 23) | |
| CDR2 | GISNRFS |
| (SEQ ID No. 24) | |
| CDR3 | LQGTHQPYT. |

Inotuzumab, Binds to an Epitope on Ig Domain 7 of CD22.

The antigen-binding domain may have one or more mutations (substitutions, additions or deletions) in one or more of the CDR sequences provided that the resultant molecule retains the capacity to bind CD22. For example, each CDR may comprise one, two or three mutations compared to the sequences given above. The mutations may be in CDR1 or 2, or the light chain CDRs, which are often less critical for antigen binding. The antigen binding domain may comprise the VH and/or VL from Inotuzumab, which are given above as SEQ ID Nos. 25 and 26 respectively or a variant thereof with, for example 80%, 90% or 95% identity which retains the capacity to bind CD22.

SEQ ID No. 25: VH sequence
EVQLVQSGAEVKKPGASVKVSCKASGYRFTNYWIHWVRQAPGQGLEWIGG
INPGN 35NYATYRRKFQGRVTMTADTSTSTVYMELSSLRSEDTAVYYCT
REGYGNYGAWFAYWGQGTLVTVSS
SEQ ID No. 26: VL sequence
DVQVTQSPSSLSASVGDRVTITCRSSQSLANSYGNTFLSWYLHKPGKAPQ
LLIYGISNRFSGVPDRFSGSGSGTDFTLTISSLQPEDFATYYCLQGTHQP
YTFGQGTKVEIK

CD22-Antigen Binding Domain: CD22ALAb

An anti-CD22 CAR which based on the CD22 binder CD22ALAb has improved properties compared to a known anti-CD22 CAR which comprises the binder m971 (WO2016/102965). The CD22 binder CD22ALAb has the CDRs and VH/VL regions identified below.

Where the first and/or second bind CD22, the antigen binding domain which may be derived from CD22ALAb, which has a heavy chain variable region (VH) having complementarity determining regions (CDRs) with the following sequences:

| (SEQ ID No. 27) | |
|---|---|
| CDR1 | NYWIN; |
| (SEQ ID No. 28) | |
| CDR2 | NIYPSDSFTNYNQKFKD |
| (SEQ ID No. 29) | |
| CDR3 | DTQERSWYFDV; |

a light chain variable region (VL) having CDRs with the following sequences:

| (SEQ ID No. 30) | |
|---|---|
| CDR1 | RSSQSLVHSNGNTYLH; |
| (SEQ ID No. 31) | |
| CDR2 | KVSNRFS |
| (SEQ ID No. 32) | |
| CDR3 | SQSTHVPWT. |

It may be possible to introduce one or more mutations (substitutions, additions or deletions) into the or each CDR without negatively affecting CD22-binding activity. Each CDR may, for example, have one, two or three amino acid mutations.

The first and/or second CAR of the cell of the present invention may comprise one of the following amino acid sequences:

(Murine CD22ALAb scFv sequence)
SEQ ID No. 33
QVQLQQPGAELVRPGASVKLSCKASGYTFTNYWINWVKQRPGQGLEWIGN
IYPSDSFTNYNQKFKDKATLTVDKSSSTAYMQLSSPTSEDSAVYYCTRDT
QERSWYFDVWGAGTTVTVSSDVVMTQTPLSLPVSLGDQASISCRSSQSLV
HSNGNTYLHWYLQKPGQSPKWYKVSNRFSGVPDRFSGSGSGTDFTLKISR
VEAEDLGLYFCSQSTHVPWTFGGGTKLEIK
(Humanised CD22ALAb scFv sequence)
SEQ ID No. 34
EVQLVESGAEVKKPGSSVKVSCKASGYTFTNYWINWVRQAPGQGLEWIGN
IYPSDSFTNYNQKFKDRATLTVDKSTSTAYLELRNLRSDDTAVYYCTRDT
QERSWYFDVWGQGTLVTVSSDIVMTQSPATLSVSPGERATLSCRSSQSLV
HSNGNTYLHWYQQKPGQAPRLLIYKVSNRFSGVPARFSGSGSGVEFTLTI
SSLQSEDFAVYYCSQSTHVPWTFGQGTRLEIK

The scFv may be in a VH-VL orientation (as shown in SEQ ID Nos 33 and 34) or a VL-VH orientation.

The first and/or second CAR of the cell of the present invention may comprise one of the following VH sequences:

(Murine CD22ALAb VH sequence)
SEQ ID No. 35
QVQLQQPGAELVRPGASVKLSCKASGYTFTNYWINWVKQRPGQGLEWIGN
IYPSDSFTNYNQKFKDKATLTVDKSSSTAYMQLSSPTSEDSAVYYCTRDT
QERSWYFDVWGAGTTVTVSS
(Humanised CD22ALAb VH sequence)
SEQ ID No. 36
EVQLVESGAEVKKPGSSVKVSCKASGYTFTNYWINWVRQAPGQGLEWIGI
YPSDSFTNYNQKFKDRATLTVDKSTSTAYLELRNLRSDDTAVYYCTRDNT
QERSWYFDVWGQGTLVTVSS

The first and/or second CAR of the cell of the present invention may comprise one of the following VL sequences:

(Murine CD22ALAb VL sequence)
SEQ ID No. 37
DVVMTQTPLSLPVSLGDQASISCRSSQSLVHSNGNTYLHWYLQKPGQSPK
WYKVSNRFSGVPDRFSGSGSGTDFTLKISRVEAEDLGLYFCSQSTHVPWT
FGGGTKLEIK
(Humanised CD22ALAb VL sequence)
SEQ ID No. 38
DIVMTQSPATLSVSPGERATLSCRSSQSLVHSNGNTYLHWYQQKPGQAPR
LLIYKVSNRFSGVPARFSGSGSGVEFTLTISSLQSEDFAVYYCSQSTHVP
WTFGQGTRLEIK

The first CAR and a second CAR of the cell of the present invention may comprise a variant of a sequence shown as SEQ ID No. 19, 20, 21, 22, 23, 24, 25, 26, 27, 28, 29, 30, 31, 32, 33, 34, 35, 36, 37, or 38, having at least 80, 85, 90, 95, 98 or 99% sequence identity, provided that the variant sequence of the first CAR and the second CAR retain the capacity to bind CD22 (when in conjunction with a complementary VL or VH domain, if appropriate) with either CD22 antigen binding domains derived from Inotuzumab or CD22ALAb.

The percentage identity between two polypeptide sequences may be readily determined by programs such as BLAST, which is freely available at http://blast.ncbi.nim.nih.gov.

In one embodiment of the invention, the first CAR of the cell comprises the antigen binding domain derived from Inotuzumab, and the second CAR of the cell comprises an antigen binding domain CD22ALab.

In another embodiment, the first CAR of the cell comprises the antigen binding domain derived from CD22ALab and the second CAR of the cell comprises the antigen binding domain derived from Inotuzumab.

In an alternative embodiment, both the first CAR and the second CAR comprise an antigen binding domain derived from either Inomuzumab or the antigen binding domain CD22ALab.

Other anti-CD22 antibodies are known, such as the mouse anti-human CD22 antibodies 1D9-3, 3B4-13, 7G6-6, 6C4-6, 4D9-12, 5H4-9, 10C1-D9, 15G7-2, 2B12-8, 2C4-4 and 3E10-7. Table 2 summarises the, VH, VL and CDR sequences (in bold and underlined) and the position of the target epitope on CD22 for each antibody.

TABLE 2

| | | | Position |
|---|---|---|---|
| | | | of epitope |
| Antibody | VH | VL | on CD22 |
| 1D9-3 | EVQLVESGGGLVQPKGSLKLSCAASGF | DIVMTQSQKFMSTSVGDRVSITC | Domain 1 |
| | TFNTYAMHWVRQAPGKGLEWVARIRSK | KASQNVRTAVAWYQQKPGQSPKA | and 2 |
| | SSNYATYYADSVKDRFTISRDDSQSML | LIYLASNRHTGVPDRFTGSGSGT | |
| | YLQMNNLKTEDTAMYYCVVDYLYAMDY | DFTLTISNVQSEDLADYFCLQHW | |
| | WGQGTSVTVSS | NYPFTFGSGTKLEIK | |
| | (SEQ ID No. 58) | (SEQ ID No. 59) | |
| 3B4-13 | QVQLQQSGAELVRPGASVTLSCKASGY | QAVVTQESALTTSPGETVTLTCR | Domain 1 |
| | TFTDYEMHWVKQTPVHGLEWIGAIDPE | SSAGAVTTSNYANWVQEKPDHLF | and 2 |
| | TGATAYNQKFKGKAILTADKSSSTAYM | TGLIGGTNNRAPGVPARFSGSLI | |
| | DLRSLTSEDSAVYYCTRYDYGSSPWFA | GDKAALTITGAQTEDEAIYFCAL | |
| | YWGQGTLVTVSA | WNSNHWVFGGGTKLTVL | |
| | (SEQ ID No. 60) | (SEQ ID No. 61) | |
| 7G6-6 | QVQLQQPGAELVMPGASVKLSCKASGY | DIVMSQSPSSLAVSVGEKVTMSC | Domain 1 |
| | TFTSYWMHWVKQRPGQGLEWIGEIDPS | KSSQSLLYSSNQKNYLAWYQQKP | and 2 |
| | DSYTNYNQKFKGKATLTVDKSSSTAYM | GQSPKWYWASTRESGVPDRFTGS | |
| | QLSSLTSEDSAVYYCARGYYGSSSFDY | GSGTDFTLTISSVKAEDLAVYYC | |
| | WGQGTTLTVSS | QQYYSYTFGGGTKLEIK | |
| | (SEQ ID No. 62) | (SEQ ID No. 63) | |
| 6C4-6 | QVQLKESGPGLVAPSQSLSITCTVSGF | DIQMTQSPASLSASVGETVTITC | Domain 3 |
| | SLTSYGVHWVRQPPGKGLEWLVVIWSD | RASENIYSYLAWYQQKQGKSPQL | |
| | GSTTYNSALKSRLSISKDNSKSQVFLK | LVYNAKTLAEGVPSRFSGSGSGT | |
| | MNSLQTDDTAMYYCARHADDYGFAWFA | QFSLKINSLQPEDFGSYYCQHHY | |
| | YWGQGTLVTVSA | GTPPTFGGGTKLEIK | |
| | (SEQ ID No. 64) | (SEQ ID No. 65) | |
| 4D9-12 | EFQLQQSGPELVKPGASVKISCKASGY | DIQMTQSPSSLSASLGERVSLTC | Domain 4 |
| | SFTDYNMNWVKQSNGKSLEWIGVINPN | RASQEISGYLSWLQQKPDGTIKR | |
| | YGTTSYNQKFKGKATLTVDQSSSTAYM | LIYAASTLDSGVPKRFSGSRSGS | |
| | QLNSLTSEDSAVYYCARSSTTVVDWYF | DYSLTISSLESEDFADYYCLQYA | |
| | DVWGTGTTVTVSS | SYPFTFGSGTKLEIK | |
| | (SEQ ID No. 66) | (SEQ ID No. 67) | |
| 5H4-9 | QVQVQQPGAELVRPGTSVKLSCKASGY | DVVMTQTPLSLPVSLGDQASISC | Domain 4 |
| | TFTRYWMYWVKQRPGQGLEWIGVIDPS | RSSQSLVHSNGNTYLHWYLQKPG | |
| | DNFTYYNQKFKGKATLTVDTSSSTAYM | QSPKLLIYKVSNRFSGVPDRFSG | |
| | QLSSLTSEDSAVYYCARGYGSSYVGYW | SGSGTDFTLKISRVEAEDLGVYF | |
| | GQGTTLTVSS | CSQSTHVPPWTFGGGTKLEIK | |
| | (SEQ ID No. 68) | (SEQ ID No. 69) | |
| 10C1-D9 | QVTLKESGPGILQSSQTLSLTCSFSGF | DIQMTQTTSSLSASLGDRVTISC | Domain 4 |
| | SLSTSDMGVSWIRQPSGKGLEWLAHIY | RASQDISNYLNWYQQKPDGTVKL | |
| | WDDDKRYNPSLKSRLTISKDASRNQVF | LIYYTSRLHSGVPSRFSGSGSGT | |
| | LKIATVDTADTATYYCARSPWIYYGHY | DYSLTISNLEQEDIATYFCQQGN | |
| | WCFDVWGTGTTVTVSS | TLPFTFGSGTKLEIK | |
| | (SEQ ID No. 70) | (SEQ ID No. 71) | |
| 15G7-2 | QVQLQQSGAELVKPGASVKLSCKASGY | QIVLTQSPAIMSASPGEKVTMTC | Domain 4 |
| | TFTEYTIHWVKQRSGQGLEWIGWFYPG | SASSSVSYMYWYQQKPGSSPRLL | |
| | SGSIKYNEKFKDKATLTADKSSSTVYM | IYDTSNLASGVPVRFSGSGSGTS | |
| | ELSRLTSEDSAVYFCARHGDGYYLPPY | YSLTISRMEAEDAATYYCQQWSS | |
| | YFDYWGQGTTLTVSS | YPLTFGAGTKLELK | |
| | (SEQ ID No. 72) | (SEQ ID No. 73) | |
| 2B12-8 | QVQLQQSGAELARPGASVKLSCKASGY | DIVLTQSPATLSVTPGDSVSLSC | Domain 4 |
| | IFTSYGISWVKQRTGQGLEWIGEIYPR | RASQSISTNLHWYQQKSHASPRL | |
| | SGNTYYNEKFKGKATLTADKSSSTAYM | LIKYASQSVSGIPSRFSGSGSGT | |
| | ELRSLTSEDSAVYFCARPIYYGSREGF | DFTLSINSVETEDFGIFFCQQSY | |
| | DYWGQGTTLTVSS | SWPYTFGGGTKLEIK | |
| | (SEQ ID No. 74) | (SEQ ID No. 75) | |
| 2C4-4 | QVQLQQPGAELVMPGASVKLSCKASGY | DVLMTQTPLSLPVSLGDQASISC | Domain 5- |
| | TFTSYWMHWVKQRPGQGLEWIGEIDPS | RSSQSIVHSNGNTYLEWYLQKPG | |
| | DSYTNYNQKFKGKSTLTVDKSSSTAYI | QSPKLLIYKVSNRFSGVPDRFSG | 7 |
| | QLSSLTSEDSAVYYCARWASYRGYAMD | SESGTDFTLKISRVEAEDLGVYY | |
| | YWGQGTSVTVSS | CFQGSHVPWTFGGGTKLEIK | |
| | (SEQ ID No. 76) | (SEQ ID No. 77) | |
| 3E10-7 | EFQLQQSGPELVKPGASVKISCKASGY | DIQMTQSPSSLSASLGERVSLTC | Domain 5- |
| | SFTDYNMNWVKQSNGKSLEWIGVINPN | RASQEISGYLSWLQQKPDGTIKR | |
| | YGTTSYNQRFKGKATLTVDQSSSTAYM | LIYAASTLDSGVPKRFSGSRSGS | 7 |
| | QLNSLTSEDSAVYYCARSGLRYWYFDV | DYSLTISSLESEDFADYYCLQYA | |
| | WGTGTTVTVSS | SYPFTFGSGTKLEIK | |
| | (SEQ ID No. 78) | (SEQ ID No. 79) | |

An antigen binding domain of a chimeric receptor which binds to CD22 may comprise the VH and/or VL sequence from any of the CD22 antibodies listed in table 2, or a variant thereof which has at least 70, 80, 90 or 90% sequence identity, which variant retains the capacity to bind CD22.

Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

Where the cell of the present invention comprises two or more chimeric receptors, the spacers may be the same or different.

The spacer sequence may, for example, comprise an IgG1 Fc region, an IgG1 hinge or a CD8 stalk. The linker may alternatively comprise an alternative linker sequence which has similar length and/or domain spacing properties as an IgG1 Fc region, an IgG1 hinge or a CD8 stalk.

A human IgG1 spacer may be altered to remove Fc binding motifs.

Examples of amino acid sequences for these spacers are given below:

SEQ ID NO. 39 (hinge-CH2CH3 of human IgG1)
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
SEQ ID NO. 40 (human CD8 stalk):
TTTPAPRPPTPAPTIASQPLSLRPEACRPAAGGAVHTRGLDFACDI
SEQ ID NO. 41 (human IgG1 hinge):
AEPKSPDKTHTCPPCPKDPK

The spacer may be monomeric or dimeric. Monomeric spacers may be generated, for example, by mutation of the cysteine residue(s) responsible for disulphide bond formation (Bridgeman et al., 2010 J. Immunol. 184:6938-6949).

Where the first CAR and second CAR bind the same antigen or similar sized antigens, the spacer of the first CAR may be sufficiently different from the spacer of the second CAR in order to avoid cross-pairing but sufficiently similar to co-localise. Pairs of orthologous spacer sequences may be employed. Examples are murine and human CD8 stalks, or alternatively spacer domains which are monomeric—for instance the ectodomain of CD2.

Examples of spacer pairs which co-localise are shown in the following Table 2:

TABLE 2

| Stimulatory CAR spacer | Inhibitory CAR spacer |
|---|---|
| Human-CD8aSTK | Mouse CD8aSTK |
| Human-CD28STK | Mouse CD8aSTK |
| Human-IgG-Hinge | Human-CD3z ectodomain |
| Human-CD8aSTK | Mouse CD28STK |
| Human-CD28STK | Mouse CD28STK |
| Human-IgG-Hinge-CH2CH3 | Human-IgM-Hinge-CH2CH3CD4 |

Where the first and second CAR bind to different antigens, spacers may be chosen which give an approximately equal synapse distance between the target cell and the T-cell in the synapse.

The length of the spacers of the two CARs may be chosen such that upon binding the target cell, the intermolecular distance of:

(X+X1)≈(Y+Y1)

where:

X is the distance from the target cell membrane to the epitope on the first antigen;

X1 is the length of the first CAR;

Y is the distance from the target cell membrane to the epitope on the second antigen;

Y1 is the length of the second CAR.

Transmembrane Domain

The transmembrane domain is the sequence of the CAR that spans the membrane. A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may, for example, be derived from CD28, which gives good receptor stability.

Activating Endodomain

The activating endodomain is the signal-transmission portion of a classical CAR. After antigen recognition by the antigen binding domain, individual CAR molecules cluster, native CD45 and CD148 are excluded from the synapse and a signal is transmitted to the cell.

The activating endodomain of the second CAR of the cell of the first aspect of the invention may comprise intracellular signalling domain. In an alternative embodiment, the activating endodomain of the present CAR may be capable of interacting with an intracellular signalling molecule which is present in the cytoplasm, leading to signalling.

The intracellular signalling domain or separate intracellular signalling molecule may be or comprise a T cell signalling domain.

The activating endodomain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/I. Two of these signatures are typically separated by between 6 and 8 amino acids in the tail of the molecule (YxxL/Ix₍₆₋₈₎YxxL/I).

ITAMs are important for signal transduction in immune cells. Hence, they are found in the tails of important cell signaling molecules such as the CD3 and ξ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signaling pathways of the cell.

The most commonly used signalling domain component is that of CD3-ξ endodomain, which contains three ITAMs. This transmits an activation signal to the T cell after antigen is bound. CD3-ξ may not provide a fully competent activation signal and additional co-stimulatory signalling may be needed. For example, 4-1BB (also known as CD137) can be used with CD3-ξ, or CD28 and OX40 can be used with CD3-ξ to transmit a proliferative/survival signal.

The activating endodomain of the second CAR of the cell of the first aspect of the invention may comprise the CD3-ξ endodomain alone, the CD3-ξ endodomain in combination with one or more co-stimulatory domains selected from 4-1BB, CD28 or OX40 endodomain, and/or a combination of some or all of 4-1BB, CD28 or OX40.

The endodomain may comprise one or more of the following: an ICOS endodomain, a CD27 endodomain, a BTLA endodomain, a CD30 endodomain, a GITR endodomain and an HVEM endodomain.

The endomain may comprise the sequence shown as SEQ ID NO 42 to 45 or a variant thereof having at least 80% sequence identity.

CD3-ζ endodomain
SEQ ID NO 42
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
4-1BB and CD3-ζ endodomains
SEQ ID NO 43
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCELRVKFSRSADAPAYQQGQNQLYNELNLGRREEYDVLDKRRGRDPEM
GGKPQRRKNPQEGLYNELQKDKMAEAYSEIGMKGERRRGKGHDGLYQGLS
TATKDTYDALHMQALPPR
CD28 and CD3-ζ endodomains
SEQ ID NO 44
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR
CD28, OX40 and CD3-ζ endodomains
SEQ ID NO 45
SKRSRLLHSDYMNMTPRRPGPTRKHYQPYAPPRDFAAYRSRDQRLPPDAH
KPPGGGSFRTPIQEEQADAHSTLAKIRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR

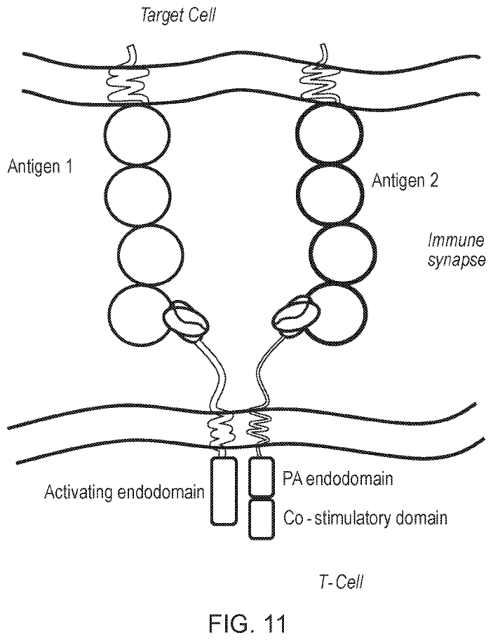

The endodomain of the first CAR of the present invention may comprise a phosphorylation amplifying domain and a co-stimulatory domain, for example as illustrated in FIG. 11. The co-stimulatory domain may, for example, be selected from one of the following endodomains: CD28, 4-1BB, OX40, ICOS, CD27, BTLA, CD30, GITR and HVEM.

Nucleic Acid Construct

In one aspect, the present invention provides a nucleic acid construct which comprises two or more nucleic acid sequences. The nucleic acid construct may comprise a first nucleic acid sequence encoding a first CAR and a second nucleic acid sequence encoding a second CAR.

For example, the nucleic acid construct may comprise:

(a) a first nucleic acid sequence encoding an antigen-binding domain, a spacer domain, a transmembrane domain and a phosphorylating amplifying domain of the first CAR and

(b) a second nucleic acid sequence encoding an antigen-binding domain, a spacer domain, a transmembrane domain, and an activating endodomain of the second CAR.

The nucleic acid construct may have the following structure:

AgB1-spacer1-TM 1-Pa-coexpr-AbB2-spacer2-TM2-endo

in which, AgB1 is a nucleic acid sequence encoding an antigen-binding domain of the first CAR;

spacer 1 is a nucleic acid sequence encoding a spacer of the first CAR;

TM1 is a nucleic acid sequence encoding a transmembrane domain of the first CAR;

Pa is a nucleic acid sequence encoding the phosphorylation amplifying domain of the first CAR;

coexpr is a nucleic acid sequence enabling co-expression of both CAR;

AgB2 is a nucleic acid sequence encoding an antigen-binding domain of the second CAR;

spacer 2 is a nucleic acid sequence encoding a spacer of the second CAR;

TM2 is a nucleic acid sequence encoding a transmembrane domain of the second CAR;

Endo is a nucleic acid sequence encoding the activating endodomain of the second CAR;

in which nucleic acid construct, when expressed in a T cell, encodes a polypeptide which is cleaved at the cleavage site such that the first and second CARs are co-expressed at the T cell surface.

Sequences encoding the first and second CARs may be in either order on the nucleic acid construct.

The activating endodomain may comprise an intracellular cell signalling domain or may associate intracellularly with a separate cell signalling domain.

The first CAR may comprise a co-stimulatory domain in addition to the phosphorylation amplifying domain. In this embodiment, the endodomain of the first CAR may comprise a CD3zeta endodomain alone, i.e. it may lack a co-stimulatory domain.

In the structure above, “coexpr” is a nucleic acid sequence enabling co-expression of both first and second CARs. It may be a sequence encoding a cleavage site, such that the nucleic acid construct produces comprises two or more CARs joined by a cleavage site(s). The cleavage site may be self-cleaving, such that when the polypeptide is produced, it is immediately cleaved into individual peptides without the need for any external cleavage activity.

The cleavage site may be any sequence which enables the first and second CARs to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above. The cleavage site may comprise one of these 2A-like sequences, such as:

(SEQ ID NO 46)
YHADYYKQRLIHDVEMNPGP
(SEQ ID NO 47)
HYAGYFADLLIHDIETNPGP
(SEQ ID NO 48)
QCTNYALLKLAGDVESNPGP
(SEQ ID NO 49)
ATNFSLLKQAGDVEENPGP
(SEQ ID NO 50)
AARQMLLLLSGDVETNPGP
(SEQ ID NO 51)
RAEGRGSLLTCGDVEENPGP
(SEQ ID NO 52)
TRAEIEDELIRAGIESNPGP
(SEQ ID NO 53)
TRAEIEDELIRADIESNPGP
(SEQ ID NO 54)
AKFQIDKILISGDVELNPGP
(SEQ ID NO 55)
SSIIRTKMLVSGDVEENPGP
(SEQ ID NO 56)
CDAQRQKLLLSGDIEQNPGP
(SEQ ID NO 57)
YPIDFGGFLVKADSEFNPGP

The cleavage site may comprise the 2A-like sequence shown as SEQ ID NO 51

(RAEGRGSLLTCGDVEENPGP).

The co-expressing sequence may be an internal ribosome entry sequence (IRES). The co-expressing sequence may be an internal promoter.

The present invention also provides a kit comprising one or more nucleic acid sequence(s) encoding first and second CARs as defined above.

Vector

The present invention also provides a vector, or kit of vectors, which comprises one or more nucleic acid sequence(s) encoding a first and a second CAR as defined above. Such a vector may be used to introduce the nucleic acid sequence(s) or construct into a host cell so that it expresses a first and a second CAR.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention provides a cell which comprises a first chimeric antigen receptor (CAR) and a second CAR.

The cell may comprise a nucleic acid sequence or construct or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CDllc+) and plasmacytoid (CD123+) dendritic cells that have been activated with TSLP.

Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Trl cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

T or NK cells according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, CAR cells are generated by introducing DNA or RNA coding for first and second CARs by one of many means including transduction with a viral vector, transfection with DNA or RNA.

The cell of the present invention may be an ex vivo cell from a subject. The T (or NK) cell may be from a peripheral blood mononuclear cell (PBMC) sample. T or NK cells may be activated and/or expanded prior to being transduced with nucleic acid encoding the molecules providing the first or second CAR according to the first aspect of the invention.

The cell of the invention may be made by: (i) isolation of a cell-containing sample from a subject or other sources listed above; and (ii) transduction or transfection of the cells with one or more a nucleic acid sequence(s) encoding first and second CARs.

The cells may then by purified, for example, selected on the basis of expression of the antigen-binding domain of the antigen-binding polypeptide.

Pharmaceutical Composition

The present invention also relates to a pharmaceutical composition containing a plurality of cells of the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. The cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample from a subject;
(ii) transducing or transfecting such cells with a nucleic acid construct or first and second nucleic acid sequence or first and second vector, or vector provided by the present invention;
(iii) administering the cells from (ii) to a subject.

The cell-containing sample may be isolated from a subject or from other sources, for example as described above. The T or NK cells may be isolated from a subject's own peripheral blood (1st party), or in the setting of a haematopoietic stem cell transplant from donor peripheral blood (2nd party), or peripheral blood from an unconnected donor (3rd party).

The present invention provides a cell coexpressing a first CAR and a second CAR of the present invention for use in treating and/or preventing a disease.

The invention also relates to the use of a cell coexpressing a first CAR and a second CAR of the present invention in the manufacture of a medicament for the treatment and/or prevention of a disease.

The disease to be treated and/or prevented by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The cells of the present invention may be capable of killing target cells, such as cancer cells. The target cell may be a characterised by the presence of a cell-surface antigen (or cell-surface ligand) present on the surface of the cell. The target cell may express a high level of one or more inhibitory receptors, such as PDL1. Where a target cell expresses a high level of an inhibitory receptor such as PDL1, recognition by a T-cell would recruit inhibitory phosphatases such as PD1 to the T-cell:target-cell synapse. In these circumstances it is beneficial to increase the amount of kinase (i.e. amplify phosphorylation) in order to allow T-cell activation to occur.

An inhibitory receptor may contain one or more immunoreceptor tyrosine-based inhibitory motifs (ITIMs) and, when phosphorylated, may recruit SHP1 and/or SHP2. In NK calls, inhibitory receptors include member of the immunoglobulin superfamily and the c-type lectin superfamily. In T cell,s inhibitory receptors include programed cell death 1 (PD1) and cytotoxic T lymphocyte-associated Antigen 4 (CTLA-4), IM3, LAG3, CD160, BTLA, and 2B4.

Where the target cell is a malignant cell, it may express the inhibitory receptor at a level greater than the mean level of expression from an equivalent non-malignant cell.

The target cell may express the inhibitory receptor at a level which would mean that a cell expressing a classical CAR is not significantly activated upon recognition of the target cell, but a cell expressing a CAR system of the cell of the present invention (which comprises a CAR with a phosphorylation amplification domain) is activated upon recognition of the target cell.

The cells and pharmaceutical compositions of present invention may be for use in the treatment and/or prevention of the diseases described above.

The cells and pharmaceutical compositions of present invention may be for use in any of the methods described above.

Further Aspects of the Invention

The eleventh aspect of the invention further provides a chimeric antigen receptor (CAR) comprising:

a. an antigen binding domain,

b. a transmembrane domain,

c. a phosphorylation amplifying domain,

d. and an activating endodomain,

wherein the phosphorylation amplifying domain comprises a tyrosine kinase domain of Fyn.

The activating endodomain of the CAR of the eleventh aspect of the invention may comprise CD3-ξ.

The activating endodomain of the CAR of the eleventh aspect of the invention may comprise CD3-ξ and one or more co-stimulatory domain(s) 4-1BB, CD28 and/or OX40.

The activating endodomain of the CAR of the eleventh aspect of the invention may comprise CD3-ξ and the co-stimulatory domain 4-1BB.

The antigen binding domain of the CAR of the eleventh aspect of the invention may bind to any antigen associated with a disease phenotype, preferably selected from the tumour-associated antigens disclosed in table 1.

The antigen binding domain of the CAR of the eleventh aspect of the invention may bind to CD22.

The antigen binding domain of the CAR of the eleventh aspect of the invention may bind to ROR1.

In a twelfth aspect, the invention relates to a nucleic acid sequence encoding the CAR of the eleventh aspect of the invention. The nucleic acid sequence may have the following structure:

AgB-spacer-TM-endo-Pa

or

AgB-spacer-TM-Pa-endo

in which

AgB is a nucleic acid sequence encoding an antigen-binding domain of the CAR;

Spacer is a nucleic acid sequence encoding a spacer of the CAR;

TM is a nucleic acid sequence encoding a transmembrane domain of the CAR;

Endo is a nucleic acid sequence encoding the activating endodomain of the CAR;

Pa is a nucleic acid sequence encoding the phosphorylation amplifying domain of the CAR;

wherein the Pa comprises a tyrosine kinase domain of Fyn; and

which nucleic acid sequence encodes a polypeptide which is then expressed at a cell surface.

In a thirteenth aspect, the present invention provides a vector comprising the nucleic acid sequence of the twelfth aspect of the invention.

The vector may, for example, be a retroviral vector or a lentiviral vector or a transposon.

In a fourteenth aspect, the present invention provides a cell comprising a CAR of the eleventh aspect of the invention.

In a fifteenth aspect, the present invention provides a pharmaceutical composition comprising a plurality of cells of the fourteenth aspect of the invention.

In a sixteenth aspect, the present invention provides a method for treating and/or preventing a disease, which comprises the step of administering a pharmaceutical composition according to the fifteenth aspect of the invention to a subject.

The method may comprise the following steps:
(i) isolation of a cell-containing sample from a subject;
(ii) transduction or transfection of the cells with the nucleic acid sequence encoding the CAR of the twelfth aspect of the invention or the vector of the thirteenth aspect of the invention; and
(iii) administering the cells from (ii) a/to the subject.

The disease may be cancer. The cancer may be a B cell malignancy.

In a seventeenth aspect, the present invention relates to a pharmaceutical composition according to the fourteenth aspect of the invention for use in treating and/or preventing a disease.

In an eighteenth aspect, the present invention provides the use of a CAR according to the eleventh aspect of the invention in the manufacture of a medicament for treating and/or preventing a disease.

The above paragraphs relating to the cells, nucleic acids, vectors, kits, compositions and methods of the first to tenth aspects of the invention, and components thereof, also apply to the corresponding components of the eleventh to eighteenth aspects of the invention.

The invention will now be described by way of Examples, which are meant to serve to assist one of ordinary skill in the art in carrying out the invention and are not intended in any way to limit the scope of the invention.

### EXAMPLES

A series of read-outs were measured to evaluate the sensitivity of various CAR constructs to a target antigen. In particular, cytotoxicity, proliferation and cytokine production were measured.

### Example 1—FACs-Based Killing (FBK)

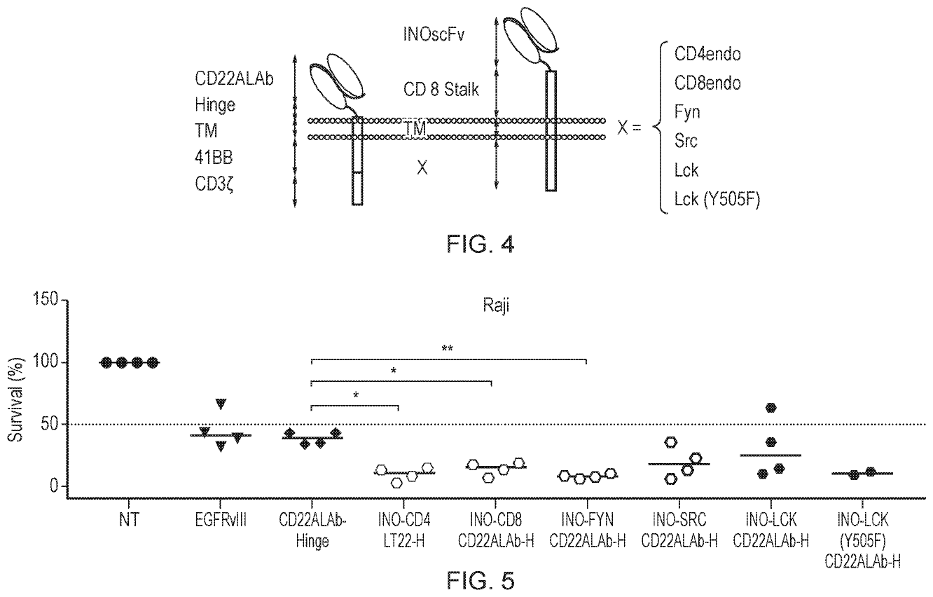

A panel of CARs was created with and without phosphorylation amplifying endodomains and their cytotoxic capability was compared. The CAR system tested comprised a first CAR comprising an CD22 antigen binding domain derived Inotuzumab (INO) and a second CAR with an CD22ALAb antigen binding domain CAR (FIG. 4). The Inotuzumab scFv tested was the clone g5/44. The phosphorylation amplifying CARs tested comprised the Inotuzumab scFv, a CD8stalk spacer, a transmembrane domain, and the intracellular domain comprising a phosphorylating amplifying endodomain comprising the tyrosine kinase domain of Fyn, Src, Lck or Lck mutant Y505F or an intracellular domain of CD4 or CD8 coreceptor. The activating CARs tested, as shown in FIG. 4, comprised a hinge spacer, a transmembrane domain and an activating endodomain comprising CD3, and 4-1BB.

Seven days after the thawing of PBMCs, the culture was depleted of CD56 NK cells to reduce background cytotoxicity. On the eighth day, the T-cells were co-cultured with the target cells at a ratio 1:1. The assay was carried out in a 96-well plate in 0.2 ml total volume using 5×10⁴ transduced T-cells per well and an equal number of target cells. The co-cultures are set up after being normalised for the transduction efficiency. The FBK was carried out after 72 h of incubation.

The results of the FBK are shown in FIG. 5. It is clear the cells co-expressing a first CAR comprising a phosphorylating amplifying (Pa) endodomain with a second CAR comprising an activating endodomain are superior. For example, the non-transduced cells, the EGFRvIII or the CD22ALAb-Hinge CAR, which lack a phosphorylating amplifying (Pa) endodomain, show significantly higher overall cell survival than those CAR constructs comprising a phosphorylating amplifying (Pa) endodomain.

The results show that a cell coexpressing a first CAR comprising a Fyn or Lck (Y505F mutant) phosphorylating amplifying domain achieved the best FACs based killing where on average the least percentage of Raji cells survived.

### Example 2—Proliferation Assay (PA)

Proliferation is a key feature of CAR-mediated responses which is measured to test the efficacy of a CAR alongside cytotoxicity and cytokine secretion. Although 1^{st} generation CARs display good levels of cytotoxicity, they do not display good proliferative responses in vitro and fail to persist well in vivo. Proliferation is enhanced by the inclusion of co-stimulatory domains such as CD28, OX40 or 4-1BB into the CAR endodomain.

In order to measure proliferation, CD56-depleted, the same panel of CAR-expressing T cells described in Example 1 were labelled with the dye Cell Trace Violet (CTV), a fluorescent dye which is hydrolysed and retained within the cell. It is excited by the 405 nm (violet) laser and fluorescence can be detected in the pacific blue channel. The CTV dye was reconstituted to 5 mM in DMSO. The T-cells were resuspended at 2×10⁶ cells per ml in PBS, and lul/ml of CTV was added. The T-cells were incubated the CTV for 20 minutes at 37° C. Subsequently, the cells were quenched by adding 5V of complete media. After a 5 minutes incubation, the T-cells were washed and resuspended in 2 ml of complete media. An additional 10 minute incubation at room temperature allowed the occurrence of acetate hydrolysis and retention of the dye.

Labelled T-cells were co-cultured with antigen-expressing or antigen-negative target cells for seven days. The assay was carried out in a 96-well plate in 0.2 ml total volume using 5×10⁴ transduced T-cells per well and an equal number of target cells (ratio 1:1). At the day seven time point, the T-cells were analysed by flow cytometry to measure the dilution of the CTV which occurs as the T-cells divide. The number of T-cells present at the end of the co-culture was calculated, and expressed as a fold of proliferation compared to the input number of T cells.

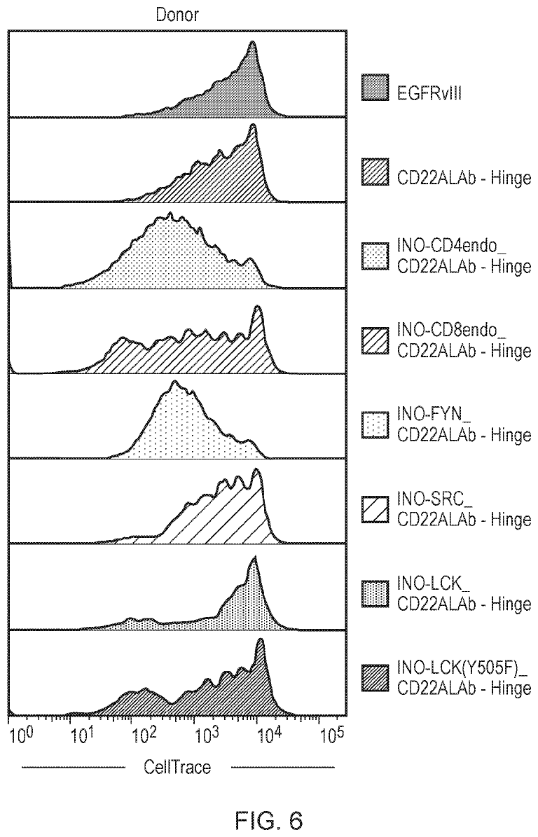

FIG. 6 shows that CAR constructs comprising a phosphorylating amplifying endodomain demonstrate increased proliferation compared to constructs lacking the phosphorylating amplifying endodomain. In particular, the CAR construct comprising the CD4 or the Fyn phosphorylating amplifying domain show the largest number of T-cells present at the end of the co-culture: the area under the curve in both CD4 and Fyn constructs have shifted furthest along the X-axis compared to the other constructs.

FIG. 6 shows that there was less persistence of the CAR T cell construct without a phosphorylation amplifying domain where the area under the curve is less elongated across the cell trace count line than the results for CAR T cell constructs comprising the phosphorylating amplifying domain.

### Example 3—Cytokine Bead Array (CBA)

Typically, immune cells detect major histocompatibility complex (MHC) presented on infected cell surfaces, triggering cytokine release, causing lysis or apoptosis. Cytokine production by CAR T cells can activate host immunity and represent a key element as to why these effector cells are successful. Cytokines such as IFN-γ and IL-2 from CAR cells also recruit and activate a variety of host immune cells to modulate the tumour microenvironment and disrupt tumour growth. Therefore to test the effectivity of the CAR constructs the inventors also chose to compare cytokine production.

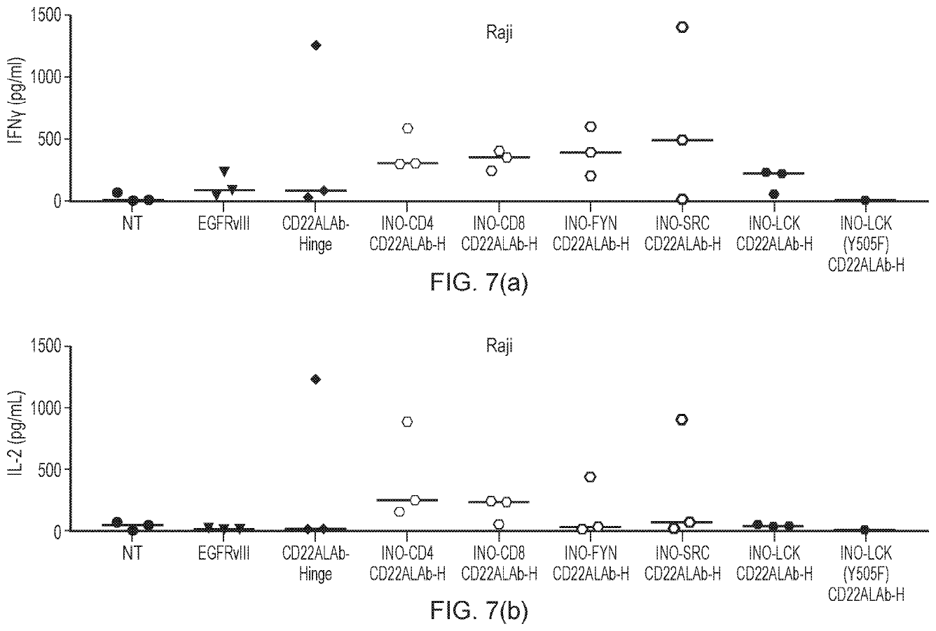

The panel of CAR constructs described in Example 1 were compared for IFN-γ secretion (FIG. 7a) and IL-2 secretion (FIG. 7b) after 72 hours co-culture with Raji target cells. Increased cytokine production was observed in the CAR constructs comprising a phosphorylating amplifying domain compared to constructs lacking a phosphorylating amplification domain. It was observed that constructs comprising CD4, CD8 coreceptors phosphorylating amplifying domains or constructs comprising Fyn phosphorylating amplifying domains presented greater cytokine production (pg/ml) than constructs comprising Lck or Lck mutant phosphorylating amplifying domains in this system.

### Example 4—Expression of BCMA on Surface of Myeloma Cells

Primary myeloma cells were isolated by performing a CD138 immunomagnetic selection on fresh bone marrow samples from Multiple myeloma patients that were known to have frank disease. These cells were stained with the BCMA specific J6MO mAb (GSK) which was conjugated to PE. At the same time, a standard of beads with known numbers of binding sites was generated using the PE Quantibrite bead kit (Becton Dickenson) as per the manufacturer's instructions. The BCMA copy number on myeloma cells was derived by correlating the mean-fluorescent intensity from the myeloma cells with the standard curve derived from the beads. It was found that the range of BCMA copy number on a myeloma cell surface is low: at 348.7-4268.4 BCMA copies per cell with a mean of 1181 and a median of 1084.9 (FIG. 8). This is considerably lower than e.g. CD19 and GD2, classic targets for CARs.

### Example 5—Investigating Killing and T-Cell Proliferation in Response to Target Cells Expressing Low Density Target Antigen

SupT1 target cells were created expressing varying levels of the target antigen CD22 at the following average antigen densities: 0, 255, 441, 634, 1090 and 78,916 copies per cell.

A panel of CARs was created and their cytotoxic capability was compared. The CAR system tested comprised; and a first CAR with an CD22ALAb antigen binding domain. The first CAR comprised a CD8 or a coiled-coil (COMP) spacer and optionally the intracellular domain of the CD4 coreceptor as phosphorylation amplifying domain. The CAR system also comprised a second CAR (activating CAR) comprising an CD22 antigen binding domain derived from the binder 10C1 and a compound 4-1BBzeta intracellular signalling domain.

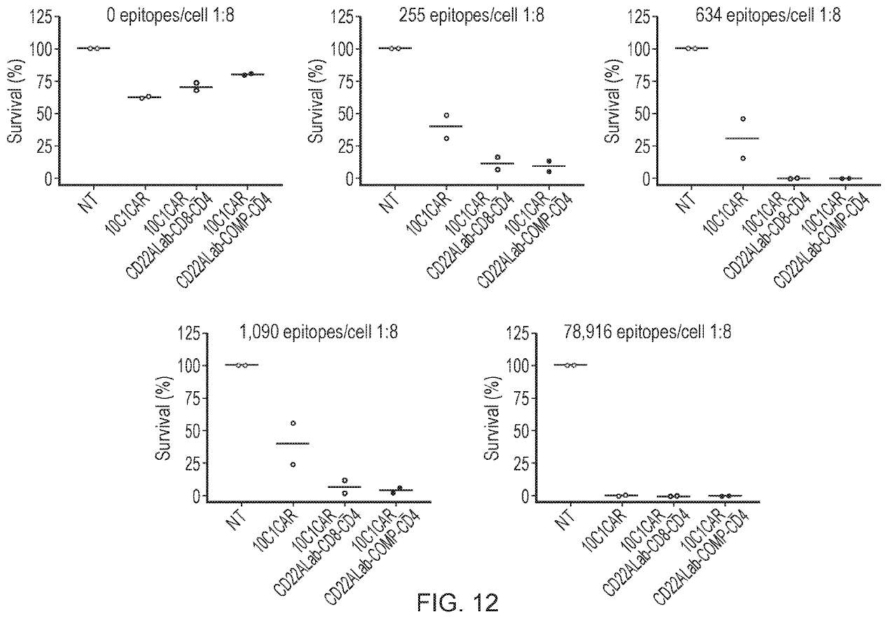

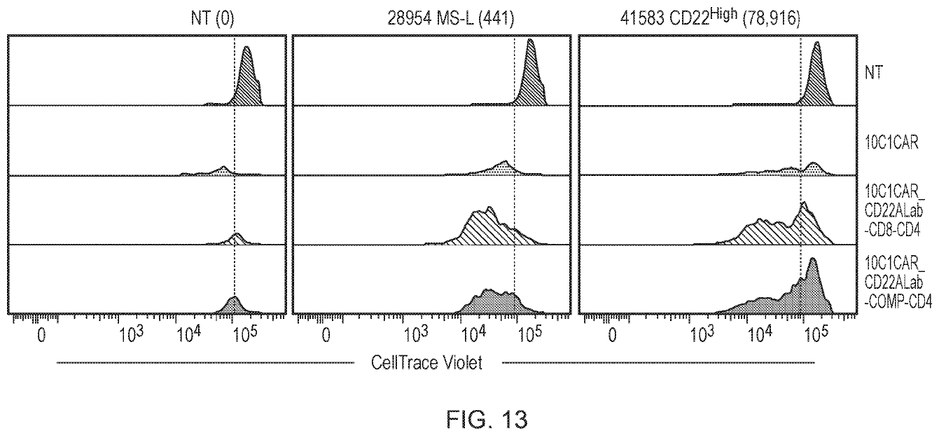

T-cells expressing these CARs were co-cultured with target cells at a 1:8 ratio for 72 hours and killing of target cells was investigated as described above. The results are shown in FIG. 12. T cells expressing either of the two constructs in which the first CAR had a CD4 endodomain (10C1CAR-CD22ALAb-CD8-CD4 and 10C1CAR-CD22ALAb-COMP-CD4) gave better killing that T cells expressing the activating CAR alone (10C1CAR) at low densities of target antigen (1090 copies per cell or below).

After 4 days co-culture, proliferation of T cells was investigated as described above. T cells expressing either of the two constructs in which the first CAR had a CD4 endodomain (10C1CAR-CD22ALAb-CD8-CD4 and 10C1CAR-CD22ALAb-COMP-CD4) showed increased proliferation compared with T-cells T cells expressing the activating CAR alone (10C1CAR) at both low (441 copies per cell) and high (78,916) target antigen densities.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1. A cell which coexpresses a first chimeric antigen receptor (CAR) and a second CAR,
wherein the first CAR comprises a phosphorylation amplifying endodomain and wherein the second CAR comprises an activating endodomain.

2. A cell according to claim 1, wherein the first CAR and the second CAR bind to different antigens.

3. A cell according to claim 1, wherein the first CAR and the second CAR bind to the same antigen.

4. A cell according to claim 3, wherein the first CAR and the second CAR bind to different epitopes.

5. A cell according to claim 3, wherein the first CAR and the second CAR bind to same epitope.

6. A cell according to claim 1 wherein the phosphorylation amplifying endodomain comprises the tyrosine kinase domain of a Src family kinase.

7. A cell according to claim 6, wherein the phosphorylation amplifying endodomain comprises the tyrosine kinase domain of Fyn, Src, Lck or a mutated Lck (Y505F).

8. A cell according to claim 6, wherein the phosphorylation amplifying endodomain comprises the tyrosine kinase domain of Fyn.

9. A cell according to claim 1 wherein the phosphorylation amplifying endodomain comprises the intracellular domain of CD4 or CD8 coreceptor.

10. A cell according to claim 1, wherein the first CAR and/or the second CAR bind to the antigen CD22.

11. A nucleic acid construct comprising a first nucleic acid sequence encoding a first chimeric antigen receptor (CAR) and a second nucleic acid sequence encoding a second CAR, wherein the first CAR comprises a phosphorylation amplifying endodomain and wherein the second CAR comprises an activating endodomain.

12. A nucleic acid construct according to claim 11, which has the following structure:
AgB1-spacer1-TM1-Pa-coexpr-AbB2-spacer2-TM2-endo
in which AgB1 is a nucleic acid sequence encoding an antigen-binding domain of the first CAR;
spacer 1 is a nucleic acid sequence encoding a spacer of the first CAR;
TM1 is a nucleic acid sequence encoding a transmembrane domain of the first CAR;
Pa is a nucleic acid sequence encoding the phosphorylation amplifying endodomain of the first CAR;
coexpr is a nucleic acid sequence enabling co-expression of both CARs;
AgB2 is a nucleic acid sequence encoding an antigen-binding domain of the second CAR;
spacer 2 is a nucleic acid sequence encoding a spacer of the second CAR;
TM2 is a nucleic acid sequence encoding a transmembrane domain of the second CAR;
Endo is a nucleic acid sequence encoding the activating endodomain of the second CAR;
which nucleic acid sequence, when expressed in a T cell, encodes a polypeptide which is cleaved such that the first and second CARs are co-expressed at the T cell surface.

13-14. (canceled)

15. A kit which comprises a first nucleic acid sequence encoding a first chimeric antigen receptor (CAR) and a second nucleic acid sequence encoding a second CAR,
wherein the first CAR comprises a phosphorylation amplifying endodomain and wherein the second CAR comprises an activating endodomain
and wherein:
(i) the first nucleic acid sequence has the following structure:
AgB1-spacer1-TM1-Pa
in which:
AgB1 is a nucleic acid sequence encoding an antigen-binding domain of the first CAR;
spacer 1 is a nucleic acid sequence encoding a spacer of the first CAR;
TM1 is a nucleic acid sequence encoding a transmembrane domain of the first CAR; and
Pa is a nucleic acid sequence encoding the phosphorylation amplifying endodomain of the first CAR; and
(ii) the second nucleic acid sequence has the following structure:
AgB2-spacer2-TM2-endo
in which:
AgB2 is a nucleic acid sequence encoding an antigen-binding domain of the second CAR;
spacer 2 is a nucleic acid sequence encoding a spacer of the second CAR;
TM2 is a nucleic acid sequence encoding a transmembrane domain of the second CAR; and
endo is a nucleic acid sequence encoding the activating endodomain of the second CAR.

16-17. (canceled)

18. A vector comprising a nucleic acid construct according to claim 11.

19. (canceled)

20. A method for making a cell according to claim 1, which comprises the step of introducing a first nucleic acid sequence encoding a first chimeric antigen receptor (CAR) and a second nucleic acid sequence encoding a second CAR into a cell, wherein the first CAR comprises a phosphorylation amplifying endodomain and wherein the second CAR comprises an activating endodomain.

21. (canceled)

22. A pharmaceutical composition comprising a plurality of cells according to claim 10.

23. A method for treating and/or preventing a disease, which comprises the step of administering a pharmaceutical composition according to claim 22 to a subject.

24. A method according to claim 22, which comprises the following steps:
(i) isolation of a cell-containing sample from a subject;
(ii) transduction or transfection of the cells with a first nucleic acid sequence encoding a first chimeric antigen receptor (CAR) and a second nucleic acid sequence encoding a second CAR into a cell, wherein the first CAR comprises a phosphorylation amplifying endodomain and wherein the second CAR comprises an activating endodomain; and
(iii) administering the cells from (ii) to the subject.

25. A method according to claim 23, wherein the disease is a cancer.

26-27. (canceled)


SEQUENCE LISTING (82 sequences)

SEQ ID NO: 1 - protein, 254 aa, Artificial Sequence
  Tyrosine kinase domain of Fyn
LQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPERALYGRFTIKSDVWSFGILLTELVTKGRVPYPGMNNREVLEQVERGYRMPCPQDCPISLHELMIHCWKKDPEERPTFEYLQSFLEDYF

SEQ ID NO: 2 - protein, 536 aa, Homo sapiens
GCVQCKDKEATKLTEERDGSLNQSSGYRYGTDPTPQHYPSFGVTSIPNYNNFHAAGGQGLTVFGGVNSSSHTGTLRTRGGTGVTLFVALYDYEARTEDDLSFHKGEKFQILNSSEGDWWEARSLTTGETGYIPSNYVAPVDSIQAEEWYFGKLGRKDAERQLLSFGNPRGTFLIRESETTKGSYSLSIRDWDDMKGDHVKHYKIRKLDNGGYYITTRAQFETLQQLVQHYSERAAGLCCRLVVPCHKGMPRLTDLSVKTKDVWEIPRESLQLIKRLGNGQFGEVWMGTWNGNTKVAIKTLKPGTMSPESFLEEAQIMKKLKHDKLVQLYAVVSEEPIYIVTEYMNKGSLLDFLKDGEGRALKLPNLVDMAAQVAAGMAYIERMNYIHRDLRSANILVGNGLICKIADFGLARLIEDNEYTARQGAKFPIKWTAPERALYGRFTIKSDVWSFGILLTELVTKGRVPYPGMNNREVLEQVERGYRMPCPQDCPISLHELMIHCWKKDPEERPTFEYLQSFLEDYFTATEPQYQPGENL

SEQ ID NO: 3 - protein, 254 aa, Artificial Sequence
  Tyrosine kinase domain of Src
LRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYF

SEQ ID NO: 4 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 5 - protein, 254 aa, Artificial Sequence
  Tyrosine kinase domain of Lck
LKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFF

SEQ ID NO: 6 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 7 - protein, 254 aa, Artificial Sequence
  Tyrosine kinase domain of Lck_Y505F
LKLVERLGAGQFGEVWMGYYNGHTKVAVRSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFF

SEQ ID NO: 8 - protein, 509 aa, Artificial Sequence
  Full length Lck_Y505F
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVRSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQFQPQP

SEQ ID NO: 9 - protein, 40 aa, Artificial Sequence
  Cytoplasmic tail of CD4
CVRCRHRRRQAERMSQIKRLLSEKKTCQCPHRFQKTCSPI

SEQ ID NO: 10 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 11 - protein, 32 aa, Artificial Sequence
  Cytoplasmic tail of CD8
LYCNHRNRRRVCKCPRPVVKSGDKPSLSARYV

SEQ ID NO: 12 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 13 - protein, 246 aa, Artificial Sequence
  daratumumab scFv sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRSGGGGSGGGGSGGGGSEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSS

SEQ ID NO: 14 - protein, 244 aa, Artificial Sequence
  anti-CD10 scFv sequence
DIVMTQSPDSLAVSLGERATINCSVSSSISSSNLHWYQQKPGQPPKLLIYGTSNLASGVPDRFSGSGSGTDFTLTISSLQAEDVAVYYCQQWSSYPLTFGQGTKVEIKRSGGGGSGGGGSGGGGSEVQLVESGGGVVQPGRSLRLSCAASGFTFSSFGMHWVRQAPGKGLEWVAYISGGSYTIYYADTVKGRFTISRDNSKNTLYLQMNSLRAEDTAVYYCARSYGNFWYFDVWGQGTTVTVSS

SEQ ID NO: 15 - protein, 135 aa, Artificial Sequence
  truncated APRIL sequence
SVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 16 - protein, 246 aa, Artificial Sequence
  anti-BCMA scFv sequence
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 17 - protein, 246 aa, Artificial Sequence
  anti-TACI scFv sequence
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYRTFGGGTKLEIKRSGGGGSGGGGSGGGGSQVTLKESGPGMLQPSQTLSLTCSFSGFSLSTFGMGVGWIRQPSGKGLEWLAHIWWDDAQYSNPALRSRLTISKDTSKNQVFLKIANVDTADTATYYCSRIHSYYSYDEGFAYWGQGTLVTVSS

SEQ ID NO: 18 - protein, 250 aa, Artificial Sequence
  anti-CD22 scFv sequence
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRSGGGGSGGGGSGGGGSEVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 19 - protein, 5 aa, Artificial Sequence
  heavy chain variable region (VH) complementarity determining region (CDR) CDR1
NYWIH

SEQ ID NO: 20 - protein, 17 aa, Artificial Sequence
  heavy chain variable region (VH) CDR2
GINPGNNYATYRRKFQG

SEQ ID NO: 21 - protein, 12 aa, Artificial Sequence
  heavy chain variable region (VH) CDR3
EGYGNYGAWFAY

SEQ ID NO: 22 - protein, 16 aa, Artificial Sequence
  light chain variable region (VL) CDR1
RSSQSLANSYGNTFLS

SEQ ID NO: 23 - protein, 7 aa, Artificial Sequence
  light chain variable region (VL) CDR2
GISNRFS

SEQ ID NO: 24 - protein, 9 aa, Artificial Sequence
  light chain variable region (VL) CDR3
LQGTHQPYT

SEQ ID NO: 25 - protein, 121 aa, Artificial Sequence
  VH sequence from Inotuzumab
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 26 - protein, 112 aa, Artificial Sequence
  VL sequence from Inotuzumab
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIK

SEQ ID NO: 27 - protein, 5 aa, Artificial Sequence
  heavy chain variable region (VH) complementarity determining region (CDR) CDR1
NYWIN

SEQ ID NO: 28 - protein, 17 aa, Artificial Sequence
  heavy chain variable region (VH) CDR2
NIYPSDSFTNYNQKFKD

SEQ ID NO: 29 - protein, 11 aa, Artificial Sequence
  heavy chain variable region (VH) CDR3
DTQERSWYFDV

SEQ ID NO: 30 - protein, 16 aa, Artificial Sequence
  light chain variable region (VL) CDR1
RSSQSLVHSNGNTYLH

SEQ ID NO: 31 - protein, 7 aa, Artificial Sequence
  light chain variable region (VL) CDR2
KVSNRFS

SEQ ID NO: 32 - protein, 9 aa, Artificial Sequence
  light chain variable region (VL) CDR3
SQSTHVPWT

SEQ ID NO: 33 - protein, 232 aa, Artificial Sequence
  Murine CD22ALAb scFv sequence
QVQLQQPGAELVRPGASVKLSCKASGYTFTNYWINWVKQRPGQGLEWIGNIYPSDSFTNYNQKFKDKATLTVDKSSSTAYMQLSSPTSEDSAVYYCTRDTQERSWYFDVWGAGTTVTVSSDVVMTQTPLSLPVSLGDQASISCRSSQSLVHSNGNTYLHWYLQKPGQSPKLLIYKVSNRFSGVPDRFSGSGSGTDFTLKISRVEAEDLGLYFCSQSTHVPWTFGGGTKLEIK

SEQ ID NO: 34 - protein, 232 aa, Artificial Sequence
  Humanised CD22ALAb scFv sequence
EVQLVESGAEVKKPGSSVKVSCKASGYTFTNYWINWVRQAPGQGLEWIGNIYPSDSFTNYNQKFKDRATLTVDKSTSTAYLELRNLRSDDTAVYYCTRDTQERSWYFDVWGQGTLVTVSSDIVMTQSPATLSVSPGERATLSCRSSQSLVHSNGNTYLHWYQQKPGQAPRLLIYKVSNRFSGVPARFSGSGSGVEFTLTISSLQSEDFAVYYCSQSTHVPWTFGQGTRLEIK

SEQ ID NO: 35 - protein, 120 aa, Artificial Sequence
  Murine CD22ALAb VH sequence
QVQLQQPGAELVRPGASVKLSCKASGYTFTNYWINWVKQRPGQGLEWIGNIYPSDSFTNYNQKFKDKATLTVDKSSSTAYMQLSSPTSEDSAVYYCTRDTQERSWYFDVWGAGTTVTVSS

SEQ ID NO: 36 - protein, 120 aa, Artificial Sequence
  Humanised CD22ALAb VH sequence
EVQLVESGAEVKKPGSSVKVSCKASGYTFTNYWINWVRQAPGQGLEWIGNIYPSDSFTNYNQKFKDRATLTVDKSTSTAYLELRNLRSDDTAVYYCTRDTQERSWYFDVWGQGTLVTVSS

SEQ ID NO: 37 - protein, 112 aa, Artificial Sequence
  Murine CD22ALAb VL sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGLYFCSQSTHVPWTFGGGTKLEIK

SEQ ID NO: 38 - protein, 112 aa, Artificial Sequence
  Humanised CD22ALAb VL sequence
DIVMTQSPATLSVSPGERATLSCRSSQSLVHSNGNTYLHWYQQKPGQAPRLLIYKVSNRFSGVPARFSGSGSGVEFTLTISSLQSEDFAVYYCSQSTHVPWTFGQGTRLEIK

SEQ ID NO: 39 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 40 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 41 - protein, 20 aa, Artificial Sequence
  spacer sequence, human IgG1 hinge
AEPKSPDKTHTCPPCPKDPK

SEQ ID NO: 42 - protein, 112 aa, Artificial Sequence
  CD3-zeta endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 368 aa, Artificial Sequence
  4-1BB and CD3-zeta endodomains
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 152 aa, Artificial Sequence
  CD28 and CD3-zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 188 aa, Artificial Sequence
  CD28, OX40 and CD3-zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 20 aa, Artificial Sequence
  cleavage site, 2A-like sequence
YHADYYKQRLIHDVEMNPGP

SEQ ID NO: 47 - protein, 20 aa, Artificial Sequence
  cleavage site, 2A-like sequence
HYAGYFADLLIHDIETNPGP

SEQ ID NO: 48 - protein, 20 aa, Artificial Sequence
  cleavage site, 2A-like sequence
QCTNYALLKLAGDVESNPGP

SEQ ID NO: 49 - protein, 19 aa, Artificial Sequence
  cleavage site, 2A-like sequence
ATNFSLLKQAGDVEENPGP

SEQ ID NO: 50 - protein, 19 aa, Artificial Sequence
  cleavage site, 2A-like sequence
AARQMLLLLSGDVETNPGP

SEQ ID NO: 51 - protein, 20 aa, Artificial Sequence
  cleavage site, 2A-like sequence
RAEGRGSLLTCGDVEENPGP

SEQ ID NO: 52 - protein, 20 aa, Artificial Sequence
  cleavage site, 2A-like sequence
TRAEIEDELIRAGIESNPGP

SEQ ID NO: 53 - protein, 20 aa, Artificial Sequence
  cleavage site, 2A-like sequence
TRAEIEDELIRADIESNPGP

SEQ ID NO: 54 - protein, 20 aa, Artificial Sequence
  cleavage site, 2A-like sequence
AKFQIDKILISGDVELNPGP

SEQ ID NO: 55 - protein, 20 aa, Artificial Sequence
  cleavage site, 2A-like sequence
SSIIRTKMLVSGDVEENPGP

SEQ ID NO: 56 - protein, 20 aa, Artificial Sequence
  cleavage site, 2A-like sequence
CDAQRQKLLLSGDIEQNPGP

SEQ ID NO: 57 - protein, 20 aa, Artificial Sequence
  cleavage site, 2A-like sequence
YPIDFGGFLVKADSEFNPGP

SEQ ID NO: 58 - protein, 119 aa, Artificial Sequence
  antibody 1D9-3, VH sequence
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVVDYLYAMDYWGQGTSVTVSS

SEQ ID NO: 59 - protein, 107 aa, Artificial Sequence
  antibody 1D9-3, VL sequence
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPFTFGSGTKLEIK

SEQ ID NO: 60 - protein, 120 aa, Artificial Sequence
  antibody 3B4-13, VH sequence
QVQLQQSGAELVRPGASVTLSCKASGYTFTDYEMHWVKQTPVHGLEWIGAIDPETGATAYNQKFKGKAILTADKSSSTAYMDLRSLTSEDSAVYYCTRYDYGSSPWFAYWGQGTLVTVSA

SEQ ID NO: 61 - protein, 109 aa, Artificial Sequence
  antibody 3B4-13, VL sequence
QAVVTQESALTTSPGETVTLTCRSSAGAVTTSNYANWVQEKPDHLFTGLIGGTNNRAPGVPARFSGSLIGDKAALTITGAQTEDEAIYFCALWNSNHWVFGGGTKLTVL

SEQ ID NO: 62 - protein, 119 aa, Artificial Sequence
  antibody 7G6-6, VH sequence
QVQLQQPGAELVMPGASVKLSCKASGYTFTSYWMHWVKQRPGQGLEWIGEIDPSDSYTNYNQKFKGKATLTVDKSSSTAYMQLSSLTSEDSAVYYCARGYYGSSSFDYWGQGTTLTVSS

SEQ ID NO: 63 - protein, 111 aa, Artificial Sequence
  antibody 7G6-6, VL sequence
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYTFGGGTKLEIK

SEQ ID NO: 64 - protein, 120 aa, Artificial Sequence
  antibody 6C4-6, VH sequence
QVQLKESGPGLVAPSQSLSITCTVSGFSLTSYGVHWVRQPPGKGLEWLVVIWSDGSTTYNSALKSRLSISKDNSKSQVFLKMNSLQTDDTAMYYCARHADDYGFAWFAYWGQGTLVTVSA

SEQ ID NO: 65 - protein, 107 aa, Artificial Sequence
  antibody 6C4-6, VL sequence
DIQMTQSPASLSASVGETVTITCRASENIYSYLAWYQQKQGKSPQLLVYNAKTLAEGVPSRFSGSGSGTQFSLKINSLQPEDFGSYYCQHHYGTPPTFGGGTKLEIK

SEQ ID NO: 66 - protein, 121 aa, Artificial Sequence
  antibody 4D9-12, VH sequence
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARSSTTVVDWYFDVWGTGTTVTVSS

SEQ ID NO: 67 - protein, 107 aa, Artificial Sequence
  antibody 4D9-12, VL sequence
DIQMTQSPSSLSASLGERVSLTCRASQEISGYLSWLQQKPDGTIKRLIYAASTLDSGVPKRFSGSRSGSDYSLTISSLESEDFADYYCLQYASYPFTFGSGTKLEIK

SEQ ID NO: 68 - protein, 118 aa, Artificial Sequence
  antibody 5H4-9, VH sequence
QVQVQQPGAELVRPGTSVKLSCKASGYTFTRYWMYWVKQRPGQGLEWIGVIDPSDNFTYYNQKFKGKATLTVDTSSSTAYMQLSSLTSEDSAVYYCARGYGSSYVGYWGQGTTLTVSS

SEQ ID NO: 69 - protein, 113 aa, Artificial Sequence
  antibody 5H4-9, VL sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIK

SEQ ID NO: 70 - protein, 124 aa, Artificial Sequence
  antibody 10C1-D9, VH sequence
QVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSS

SEQ ID NO: 71 - protein, 107 aa, Artificial Sequence
  antibody 10C1-D9, VH sequence
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 72 - protein, 123 aa, Artificial Sequence
  antibody 15G7-2, VH sequence
QVQLQQSGAELVKPGASVKLSCKASGYTFTEYTIHWVKQRSGQGLEWIGWFYPGSGSIKYNEKFKDKATLTADKSSSTVYMELSRLTSEDSAVYFCARHGDGYYLPPYYFDYWGQGTTLTVSS

SEQ ID NO: 73 - protein, 106 aa, Artificial Sequence
  antibody 15G7-2, VH sequence
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELK

SEQ ID NO: 74 - protein, 121 aa, Artificial Sequence
  antibody 2B12-8, VH sequence
QVQLQQSGAELARPGASVKLSCKASGYIFTSYGISWVKQRTGQGLEWIGEIYPRSGNTYYNEKFKGKATLTADKSSSTAYMELRSLTSEDSAVYFCARPIYYGSREGFDYWGQGTTLTVSS

SEQ ID NO: 75 - protein, 107 aa, Artificial Sequence
  antibody 2B12-8, VH sequence
DIVLTQSPATLSVTPGDSVSLSCRASQSISTNLHWYQQKSHASPRLLIKYASQSVSGIPSRFSGSGSGTDFTLSINSVETEDFGIFFCQQSYSWPYTFGGGTKLEIK

SEQ ID NO: 76 - protein, 120 aa, Artificial Sequence
  antibody 2C4-4, VH sequence
QVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSS

SEQ ID NO: 77 - protein, 112 aa, Artificial Sequence
  antibody 2C4-4, VL sequence
DVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIK

SEQ ID NO: 78 - protein, 119 aa, Artificial Sequence
  antibody 3E10-7, VH sequence
EFQLQQSGPELVKPGASVKISCKASGYSFTDYNMNWVKQSNGKSLEWIGVINPNYGTTSYNQRFKGKATLTVDQSSSTAYMQLNSLTSEDSAVYYCARSGLRYWYFDVWGTGTTVTVSS

SEQ ID NO: 79 - protein, 107 aa, Artificial Sequence; identical to SEQ ID NO: 67
  antibody 3E10-7, VL sequence

SEQ ID NO: 80 - protein, 4 aa, Artificial Sequence
  ITAM motif
  misc_feature (2)..(3): Xaa can be any naturally occurring amino acid
  MISC_FEATURE (4)..(4): Xaa may be Leu or Ile
YXXX

SEQ ID NO: 81 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 82 - protein, 7 aa, Artificial Sequence
  consensus Tobacco Etch Virus (TEV) cleavage site
ENLYFQS